(19) United States
(12) Patent Application Publication
(10) Pub. No.: US 2024/0101966 A1
(43) Pub. Date: Mar. 28, 2024

(54) End-to-End Platform for Human Pluripotent Stem Cell Manufacturing

(71) Applicant: LONZA WALKERSVILLE, Inc.

(72) Inventors: Inbar Friedrich Ben-Nun, Rockville, MD (US); Eytan Abraham, Potomac, MD (US)

(21) Appl. No.: 17/768,610

(22) PCT Filed: Nov. 20, 2020
(86) PCT No.: PCT/US2020/061450

§ 371 Date: Apr. 13, 2022

Related U.S. Application Data
(60) Provisional application No. 62/939,904, filed on Nov. 25, 2019.

(51) Int. Cl. C12N 5/074; C12M 1/00; C12M 1/06; C12M 1/26

(52) U.S. Cl. CPC C12N 5/0696; C12M 27/02; C12M 29/04; C12M 33/10; C12N 2531/00

(57) ABSTRACT

A closed, automated and scalable stirred tank bioreactor platform, capable of sustaining high fold expansion of hPSCs is provided. hPSCs are expanded in a controlled bioreactor using perfused xeno-free media. Cell harvest and concentration are performed in closed steps. The hPSCs can be cryopreserved to generate a bank of cells or further processed as needed. Cryopreserved cells can be thawed into a 2D tissue culture platform or a 3D bioreactor to initiate a new expansion phase or be differentiated to the clinically relevant cell type. The expanded hPSCs express hPSC-specific markers, have a normal karyotype and the ability to differentiate to the cells of the three germ layers. This end-to-end platform allows large expansion of high quality hPSCs that can support the required cell demand for various clinical indications.

### BACKGROUND

Stem cell technology has revolutionized regenerative medicine, ushering in a new era focused on curative therapies rather than disease management. Over the past decade, efforts toward the development and optimization of cGMP compliant, large-scale manufacturing of cell-based therapies have significantly increased. However, industrialization of stem-cell based therapies requires innovative solutions to close the gap between research and commercialization. For instance, scalable cell production platforms are needed to reliably deliver the cell quantities needed during the various stages of development and commercial supply.

Human pluripotent stem cells (hPSCs) are a key source material for generating therapeutic cell types, and successful generation of human induced pluripotent stem cells (hiPSCs) by somatic cell reprogramming has opened new avenues in regenerative medicine, disease modeling and drug development. Capable of self-renewal and pluripotency, hiPSCs derived from patients of both normal and aberrant phenotypes provide a theoretically limitless supply of clinically relevant iPSC-derived cells without existing limitations and immune-rejection. For instance, given the heart's limited to no regenerative capacity, new cardiomyocytes can instead be derived from hiPSCs by modulating developmental cues critical in embryonic development in vivo.

Essential to the successful differentiation of iPSCs to a specific cell lineage, however, includes careful consideration of the microenvironment and method with which iPSCs are maintained. While a wealth of information has been gained through the use of traditional two-dimensional (2D) culture, this system fails to generate the number of cells required in many therapies in a cost-effective manner and does not fully recapitulate in vivo conditions.

For instance, to replace the number of cells lost during a myocardial infarction, for example, approximately 1×10⁹ cells are required per patient dose. Given that 2D-based cell culture platforms are non-scalable with minimal capacity for expansion, achieving high cell densities in a 2D system would involve costly arrangements including extensive manual effort, laboratory space and personnel. These platforms also often do not possess adequate systems to control or monitor parameters such as the production of key metabolites by hiPSCs in culture. Moreover, iPSC-derived cardiomyocytes remain phenotypically immature despite a number of studies demonstrating enhanced maturation through modulation of existing methodologies.

A number of studies have demonstrated the feasibility of hPSC expansion in suspension cultures using aggregate and microcarrier (MC)-based three-dimensional (3D) culture systems. Aggregate-based 3D culture provides a more physiologically relevant microenvironment, but has been shown not only to require the small molecule, Y27632, for the survival of hPSCs, but also sequential passaging to achieve high fold expansion. Not without its own advantages, microcarrier-based culture systems facilitate larger surface area to volume ratio for scalability, provide large surface area for adhesion and growth during expansion, flexibility in using defined extracellular matrices, and allow maintenance of homogenous culture conditions.

Therefore, it would be a benefit to provide a cGMP compliant, commercially viable, scalable process to generate large numbers of high quality hPSCs. Further, it would be beneficial to provide an end-to end platform and process for hPSC expansion that solves one or more of the above problems, and/or that provides a microcarrier based expansion that uses xeno-free culture conditions. Moreover, it would be a benefit to provide an end-to-end platform and process where cells are expanded in a closed, automated and controlled single-use stirred tank bioreactor. Additionally or alternatively, it would be beneficial to provide a closed step of harvest and separation from the MCs, as well as concentrating the cells using a closed, automated centrifugation system, where the cells may be further cryopreserved. It would also be beneficial if the cryopreserved cells may also serve as the starting material, (e.g., cryopreserved hPSCs) that can be either thawed into 2D culture prior to inoculation or thawed directly into a bioreactor. It would be an additional benefit if an end-to-end platform that solved one or more of the above problems while also achieving a high expansion fold of >50 using the platform within 9-14 days of culture. Furthermore, it would be advantageous if the expanded hPSCs demonstrate high quality of self-renewal and pluripotency, and/or are capable of differentiation to all three germ layers. Additionally, it would be beneficial to provide an end-to-end platform that does not require the use of a 2D seed train.

### SUMMARY

In general, the present disclosure is directed to a process for manufacturing pluripotent stem cells. The process includes placing a plurality of microcarriers into a bioreactor, inoculating the bioreactor with pluripotent stem cells, incubating the pluripotent stem cells in the bioreactor for a period of time sufficient to yield a fold expansion of about 50 times or greater to give expanded pluripotent stem cells, concentrating the expanded pluripotent stem cells, and cryopreserving the expanded pluripotent stem cells. Further, the pluripotent stem cells are inoculated at a seeding density of about 0.2×10⁶ cells/mL or less, and the process is a closed and/or automated process.

In one aspect, the pluripotent stem cells are not passaged during incubation. Additionally or alternatively, in an aspect, the pluripotent stem cells used for inoculating the bioreactor are inoculated into the bioreactor as cryopreserved pluripotent stem cells. In a further aspect, the pluripotent stem cells are not incubated in a 2D process prior to inoculating the bioreactor.

Furthermore, in an aspect, the plurality of microcarriers have a particle size of about 125 μm or greater. Additionally or alternatively, the plurality of microcarriers are coated with a growth matrix prior to being placed in the bioreactor.

In a further aspect, the method includes a harvesting step after incubation. In an aspect, a non-enzymatic passaging solution is used to separate the microcarriers from the expanded pluripotent stem cells. Additionally or alternatively, in one aspect, after passaging with the non-enzymatic passaging solution, the pluripotent stem cells and plurality of microcarriers are run through a mesh having a mesh size sufficient to allow the pluripotent stem cells to pass through while restricting passage of the microcarriers. In one aspect, the mesh size is about 10 μm to about 100 μm.

Moreover, in a further aspect, the concentrating is performed by a continuous centrifugation device. In an aspect, a flow rate into the continuous centrifugation device is selected that allows formation of a fluidized bed in about 15 minutes or less. Furthermore, in one aspect, cell retention in the fluidized bed is about 80% or greater.

Additionally or alternatively, in an aspect, the cell retention after cryopreservation is about 70% or greater.

In one aspect, during incubation, the microcarriers and pluripotent stem cells are subject to agitation. In a further aspect, the agitation has an initial speed, and the initial speed is increased after about 1 to 5 days to a second speed. Furthermore, in an aspect, the second speed is increased after about 1 to 5 days to a third speed. Additionally or alternatively, in one aspect, the agitation has an initial speed and the initial speed is increased to a second speed when the cell density reaches about 1×10⁵ cells/cm² to about 10×10⁵ cells/cm². Moreover, in an aspect, during a first 24 hours or less after inoculation, the agitation is discontinuous agitation.

In yet another aspect, the bioreactor is a perfusion bioreactor.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1A shows a schematic representation of an end-to-end platform according to the present disclosure;

FIG. 1B is a cross sectional view of a bioreactor system in accordance with the present disclosure

FIG. 2 shows graphs of RTiPSC3B and RTiPSC4i hiPSCs growth and expansion over time;

FIG. 3 shows graphs of cell growth and expansion using small and large sized microcarriers;

FIG. 4 shows graphs of cell growth and expansion of RTiPSC4i using low cell densities;

FIG. 5 shows graphs of cell growth and expansion of RTiPSC3B using low cell densities;

FIG. 6 shows graphs of cell growth and expansion using uncoated and coated microcarriers;

FIG. 7 shows graphs of cell growth and expansion with and without microcarriers;

FIG. 8 shows graphs of consolidated cell density and fold expansion using 2D cultured cell inoculum;

FIG. 9 is images of cell-microcarrier clusters at 100× magnification;

FIG. 10 shows the monitoring of nutrient and metabolite concentrations and process parameters in 3-liter bioreactor suspension cultures of hiPSCs according to the present disclosure;

FIG. 11 shows phase contrast images of iPSCs expanded in a bioreactor according to the present disclosure at 100× magnification;

FIG. 12 shows immunofluorescence staining of iPSCs expanded in a bioreactor according to the present disclosure;

FIG. 13 is a graph of quantitative analysis of hPSC-associated markers by flow cytometry of cells expanded in a bioreactor according to the present disclosure;

FIG. 14 shows pluripotency of cells expanded in a bioreactor according to the present disclosure by immunofluorescence staining of germ layer-specific markers;

FIG. 15 shows immunofluorescence staining for lineage-specific markers for RTiPSC3B and LiPSC18R cell lines;

FIG. 16 is a graph showing the percentage of viable cells escaping the kSep chamber during fluidized bed formation according to an aspect of the present disclosure;

FIG. 17 is a graph showing the percentage of viable cells escaping the fluidized bed per run versus process time according to an aspect of the present disclosure;

FIG. 18 shows phase contrast images of single cells post concentration at 24 hours and 72 hours post plating;

FIG. 19 shows expression via immunofluorescence staining of cells expanded in a bioreactor and concentrated according to an aspect of the present disclosure;

FIG. 20 is a graph of quantitative analysis of hPSC-associated markers by flow cytometry of cells expanded in a bioreactor and concentrated according to an aspect of the present disclosure;

FIG. 21 shows pluripotency of cells expanded in a bioreactor and concentrated by directed differentiation into endoderm, neural stem cells, and cardiomyocytes, according to an aspect of the present disclosure;

FIG. 22 shows phase contrast images of cryopreserved cells 48-72 hours post thaw at 40× magnification;

FIG. 23 shows cells stained with AP staining kit at three days (vial #2) and five days post-plating;

FIG. 24 shows graphs of cell growth and fold expansion of directly thawed versus freshly inoculated cells in spinner flasks;

FIG. 25 shows graphs of cell growth and fold expansion of cells thawed into a 3-liter bioreactor according to an aspect of the present disclosure;

FIG. 26 show phase contrast images demonstrates cell growth on microcarriers on different days of the bioreactor run at 100× magnification (scale bar: 100 μm);

FIG. 27 shows iPSCs thawed into suspension and expanded in a bioreactor according to an aspect of the present disclosure have typical iPSC morphology when plated onto 2D, before and after release from the microcarriers;

FIG. 28 shows detection of hPSC-associated markers by immunofluorescence staining in cells post-harvest and concentration according to an aspect of the present disclosure;

FIG. 29 is a graph of quantitative analysis of hPSC-associated markers by flow cytometry of cells post-harvest and cells concentrated after harvest according to an aspect of the present disclosure;

FIG. 30 shows direct differentiation of iPSCs thawed into suspension, expanded in a bioreactor, and concentrated according to an aspect of the present disclosure;

FIG. 31 is a schematic representation of the experimental design for using 3D seed train as inoculum according to an aspect of the present disclosure;

FIG. 32 shows graphs of cell growth and fold expansion of LiPSC18R on microcarriers collected from a spinner flask and inoculated in a 3-liter bioreactor according to an aspect of the present disclosure;

FIG. 33 shows graphs of cell growth and fold expansion of RTiPSC3b released from microcarriers as single cells and inoculated in a 3-liter bioreactor according to an aspect of the present disclosure;

FIG. 34 shows phase contrast images of cells growing on microcarriers on different days in 3D culture at 100× Magnification (scale bar 200 μm);

FIG. 35 shows phase contrast images of colonies formed by cells expanded in a bioreactor according to an aspect of the present disclosure five days post plating at 40× magnification (Scale bar: 100 μm);

FIG. 36 shows a quality assessment of hiPSCs expanded through a 3D seed train according to an aspect of the present disclosure by immunofluorescence staining of hPSC-associated markers at 100× magnification;

FIG. 37 is a graph of quantitative analysis of hPSC-associated markers of hiPSCs expanded through a 3D seed train according to an aspect of the present disclosure;

FIG. 38 shows immunofluorescence staining of germ layer-specific markers on embryoid bodies (EBs) of hiPSCs expanded through a 3D seed train according to an aspect of the present disclosure;

FIG. 39 show a chart of a two-week cell expansion in 2D;

FIG. 40 illustrates a dip tube/perfusion line;

FIG. 41 illustrates a media feed line;

FIG. 42 illustrates a harvest line extension assembly;

FIG. 43 illustrates a gas line assembly;

FIG. 44 illustrates a gas line assembly;

FIG. 45 illustrates a 2D agitation speed characterization curve;

FIG. 46 illustrates a harvest scheme; and

FIG. 47 illustrates a flex concepts bag integrated with 65 μm mesh filter.

Repeat use of reference characters in the present specification and drawings is intended to represent the same or analogous features or elements of the present invention.

### DEFINITIONS AND ABBREVIATIONS

It is to be understood that the terminology used herein is for the purpose of describing particular embodiments only and is not intended to limit the scope of the present invention. In this specification and in the claims that follow, reference will be made to a number of terms that shall be defined to have the following meanings:

As used herein, the terms “about,” “approximately,” or “generally,” when used to modify a value, indicates that the value can be raised or lowered by 10% and remain within the disclosed embodiment.

As used herein, the term “xeno-free” refers to a medium that contains about 5% or less by weight of animal or human derived components, such as about 2% by weight or less, such as about 1% by weight or less animal or human derived components, and in one aspect, may refer to a medium completely free of animal components, human components, or both human and animal components.

Abbreviations:
hPSCs Human pluripotent stem cells
hiPSCs Human induced pluripotent stem cells
MCs Microcarriers
EBs Embryoid Bodies
BSC Bio Safety Cabinet
VVD Vessel volume per day

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to a large scale, closed system, end-to-end platform, and process related thereto, for the manufacture of human pluripotent stem cells (hPSCs) that demonstrates excellent growth, expansion, recovery, and viability. Particularly, the present disclosure has developed a microcarrier-based bioreactor suspension platform to expand hiPSCs to cell densities of >2×10⁹ cells/L using xeno-free, fully defined hPSC medium with a closed, automated process for hiPSC harvest and concentration, and extensively characterized the expanded hiPSCs. For instance, the present disclosure has found that an end-to-end platform, and related process, according to the present disclosure allows for excellent cell growth and expansion even when using lower seeding densities and/or when using larger microcarriers than previously believed. Furthermore, the present disclosure has found that an end-to-end platform, and related process, according to the present disclosure may exhibit markedly improved cell recovery and viability, even after concentration and cryopreservation. In addition, the present disclosure has unexpectedly found that cells expanded according to the present disclosure may be used to seed further expansions, allowing for 2D seed train growth to be avoided.

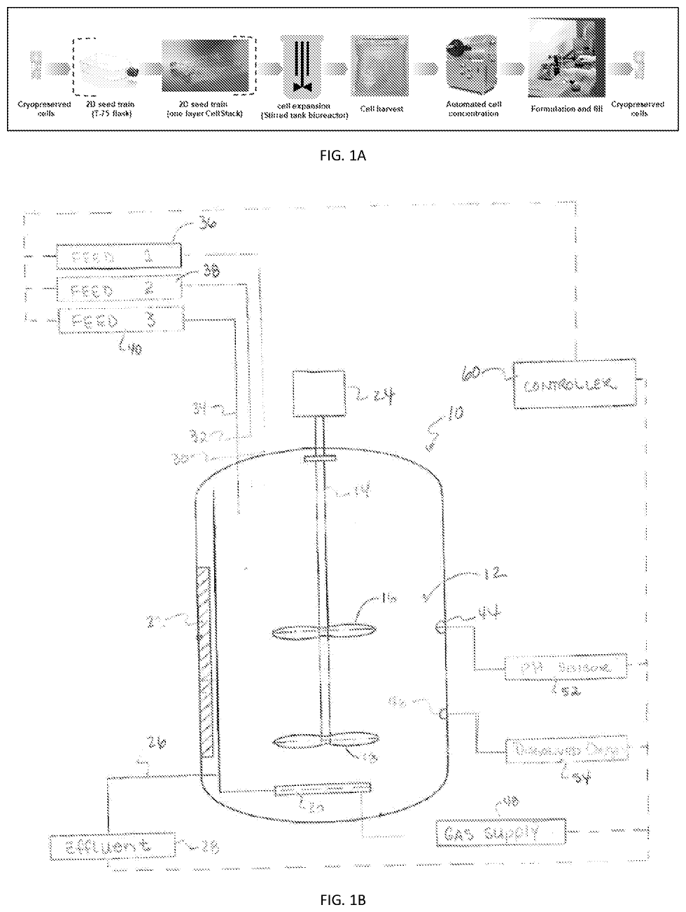

Referring first to FIG. 1A, an exemplary schematic for an end-to-end hPSC expansion platform 100 and process related thereto, will be discussed. Of course, as noted above, in one aspect, step two (104) and three (106) may be eliminated by using cryopreserved cells expanded according to the end-to-end platform 100 and process as will be described herein.

Nonetheless, in one aspect, cryopreserved cells are used to inoculate a 2D seed train flask 104. The cryopreserved cells 102 may be cryopreserved cells generally known in the art, such as cells cryopreserved in CryoStor10 and commercially available. However, in one aspect, the cryopreserved cells 102 may be cells cryopreserved 116 according to the end-to-end platform 100 and process described herein. Thus, in one aspect, the cryopreserved cells 102 are cells cryopreserved 116 in a prior batch of expanded cells.

Regardless of whether the cryopreserved cells 102 were formed according to the present disclosure or were otherwise acquired, in one aspect, the cryopreserved cells 102 can be thawed into a 2D seed train flask 104. The cells may be inoculated at a seed density of about 0.01×10⁶ cells/cm² to about 0.1×10⁶ cells/cm², such as about 0.015×10⁶ cells/cm² to about 0.05×10⁶ cells/cm², such as about 0.02×10⁶ cells/cm² to about 0.04×10⁶ cells/cm².

In one aspect, a kinase inhibitor, such as rho-associated coiled-coil containing protein kinase inhibitor (ROCKi) may be initially used with the thawed cells in the 2D seed train flask in addition to a nutrient matrix. However, after a period of time, such as about 24 hours or less, such as about 22 hours or less, such as about 20 hours or less, such as about 18 hours or less, such as about 16 hours or less, the kinase and nutrient matrix combination is replaced with an appropriate cell nutrient medium/matrix that is, in one aspect, generally free of kinase inhibitors. As used herein, a nutrient media or matrix refers to any fluid, compound, molecule, or substance that can increase the mass of a bioproduct, such as anything that may be used by an organism to live, grow or otherwise add biomass. For example, a nutrient feed can include a gas, such as oxygen or carbon dioxide that is used for respiration or any type of metabolism. Other nutrient media can include carbohydrate sources. Carbohydrate sources include complex sugars and simple sugars, such as glucose, maltose, fructose, galactose, and mixtures thereof. A nutrient media can also include an amino acid. The amino acid may comprise, glycine, alanine, valine, leucine, isoleucine, methionine, proline, phenylalanine, tryptophan, serine, threonine, asparagine, glutamine, tyrosine, cysteine, lysine, arginine, histidine, aspartic acid and glutamic acid, single stereoisomers thereof, and racemic mixtures thereof. The term “amino acid” can also refer to the known non-standard amino acids, e.g., 4-hydroxyproline, c-N,N,N-trimethyllysine, 3-methylhistidine, 5-hydroxylysine, O-phosphoserine, γ-carboxyglutamate, γ-N-acetyllysine, ω-N-methylarginine, N-acetylserine, N,N,N-trimethylalanine, N-formylmethionine, γ-aminobutyric acid, histamine, dopamine, thyroxine, citrulline, ornithine, β-cyanoalanine, homocysteine, azaserine, and S-adenosylmethionine. In some embodiments, the amino acid is glutamate, glutamine, lysine, tyrosine or valine.

The nutrient media can also contain one or more vitamins. Vitamins that may be contained in the nutrient media include group B vitamins, such as B12. Other vitamins include vitamin A, vitamin E, riboflavin, thiamine, biotin, and mixtures thereof. The nutrient media can also contain one or more fatty acids and one or more lipids. For example, a nutrient media feed may include cholesterol, steroids, and mixtures thereof. A nutrient media may also supply proteins and peptides to the bioreactor. Proteins and peptides include, for instance, albumin, transferrin, fibronectin, fetuin, and mixtures thereof. A growth medium within the present disclosure may also include growth factors and growth inhibitors, trace elements, inorganic salts, hydrolysates, and mixtures thereof. Trace elements that may be included in the growth medium include trace metals. Examples of trace metals include cobalt, nickel, and the like. For instance, and for example only, in one aspect, the nutrient medium/matrix may be L7™ hPSC matrix medium sold by Lonza.

Nonetheless, the thawed cells are seeded for growth in the 2D seed train flask 104 and maintained in the 2D seed train flask 104 until the cells reach a confluence of about 50% to about 100%, such as about 55% to about 95%, such as about 60% to about 90%, such as about 70% to about 85% confluence. For instance, in one aspect, the cells may be maintained in the 2D seed train flack 104 for about 3 days to about 9 days, such as about 4 days to about 8 days, such as about 5 days to about 7 days, in order to achieve the desired confluence and/or cell number.

After the cells have reached an appropriate confluence, the cells contained in the 2D seed train flask 104 may be passaged to a 2D seed train one-layer cell stack 106. While any passaging may be used as known in the art, in one aspect, in order to further improve cell viability and retention, a non-enzymatic cell detachment formulation may be used. For instance, in one aspect, the passaging solution may be a sodium citrate based passaging solution, such as a hypertonic sodium citrate solution, which may, in one aspect, be non-animal origin. Furthermore, in one aspect, the sodium citrate passaging solution may also include at least one salt and a liquid, such as, for example only, L7™hPSC Passaging Solution sold by Lonza.

Regardless of the passaging solution selected, the cells are placed in the 2D seed train cell stack 106 at a seeding density of about 0.01×10⁶ cells/cm² to about 0.05×10⁶ cells/cm², such as about 0.015×10⁶ cells/cm² to about 0.04×10⁶ cells/cm², such as about 0.02×10⁶ cells/cm² to about 0.03×10⁶ cells/cm². Once seeded, the cells are maintained in the 2D seed train cell stack 106 until the cells reach a confluence of about 50% to about 100%, such as about 55% to about 95%, such as about 60% to about 90%, such as about 70% to about 85% confluence. For instance, in one aspect, the cells may be maintained in the 2D seed train trays 106 for about 3 days to about 9 days, such as about 4 days to about 8 days, such as about 5 days to about 7 days, in order to achieve the desired confluence and/or cell number.

Nonetheless, after the desired cell number and/or confluence is obtained, the cells contained in the 2D seed train cell stack 106 may be harvested using a passaging solution. The passaging solution may be the same as the passaging solution discussed above or may instead use a second passaging solution. Regardless of the passaging solution selected, the harvested cells may be used to inoculate a stirred tank bioreactor 108, which may be referred to as a bioreactor herein, in order to undergo cell expansion.

In general, any suitable bioreactor may be used. The bioreactor, for instance, may comprise a fermenter, a stirred-tank reactor, an adherent bioreactor, a wave-type bioreactor, a disposable bioreactor, and the like. In the embodiment illustrated in FIG. 1B, the bioreactor 10 comprises a hollow vessel or container that includes a bioreactor volume 12 for receiving a cell culture within a fluid growth medium. As shown in FIG. 1B, the bioreactor system can further include a rotatable shaft 14 coupled to an agitator such as dual impellers 16 and 18.

The bioreactor 10 can be made from various materials. In one embodiment, for instance, the bioreactor 10 can be made from metal, such as stainless steel. Metal bioreactors are typically designed to be reused.

Alternatively, the bioreactor 10 may comprise a single use bioreactor made from a rigid polymer or a flexible polymer film. When made from a rigid polymer, for instance, the bioreactor walls can be free standing. Alternatively, the bioreactor can be made from a flexible polymer film or shape conforming material that can be liquid impermeable and can have an interior hydrophilic surface. In one aspect, the bioreactor 10 can be made from a flexible polymer film that is designed to be inserted into a rigid structure, such as a metal container for assuming a desired shape. Polymers that may be used to make the rigid vessel or flexible polymer film include polyolefin polymers, such as polypropylene and polyethylene. Alternatively, the polymer can be a polyamide. In still another embodiment, a flexible polymer film can be formed from multiple layers of different polymer materials. In one embodiment, the flexible polymer film can be gamma irradiated.

The bioreactor 10 can have any suitable volume. For instance, the volume of the bioreactor 10 can be from 0.1 mL to about 25,000 L or larger. For example, the volume 12 of the bioreactor 10 can be greater than about 0.5 L, such as greater than about 1 L, such as greater than about 2 L, such as greater than about 3 L, such as greater than about 4 L, such as greater than about 5 L, such as greater than about 6 L, such as greater than about 7 L, such as greater than about 8 L, such as greater than about 10 L, such as greater than about 12 L, such as greater than about 15 L, such as greater than about 20 L, such as greater than about 25 L, such as greater than about 30 L, such as greater than about 35 L, such as greater than about 40 L, such as greater than about 45 L. The volume of the bioreactor 10 is generally less than about 25,000 L, such as less than about 15,000 L, such as less than about 10,000 L, such as less than about 5,000 L, such as less than about 1,000 L, such as less than about 800 L, such as less than about 600 L, such as less than about 400 L, such as less than about 200 L, such as less than about 100 L, such as less than about 50 L, such as less than about 40 L, such as less than about 30 L, such as less than about 20 L, such as less than about 10 L. In one embodiment, for instance, the volume of the bioreactor can be from about 1 L to about 5 L. In an alternative embodiment, the volume of the bioreactor can be from about 25 L to about 75 L. In still another embodiment, the volume of the bioreactor can be from about 100 L to about 350 L.

In addition to the impellers 16 and 18, the bioreactor 10 can include various additional equipment, such as baffles, spargers, gas supplies, heat exchangers or thermal circulator ports, and the like which allow for the cultivation and propagation of biological cells. For example, in the embodiment illustrated in FIG. 1B, the bioreactor 10 includes a sparger 20 and a baffle 22. The sparger 20 is in fluid communication with a gas supply 48 for supplying gases to the bioreactor 10, such as carbon dioxide, oxygen and/or air. In addition, the bioreactor system can include various probes for measuring and monitoring pressure, foam, pH, dissolved oxygen, dissolved carbon dioxide, and the like.

As shown in FIG. 1B, the bioreactor 10 can include a rotatable shaft 14 attached to impellers 16 and 18. The rotatable shaft 14 can be coupled to a motor 24 for rotating the shaft 14 and the impellers 16 and 18. The impellers 16 and 18 can be made from any suitable material, such as a metal or a biocompatible polymer. Examples of impellers suitable for use in the bioreactor system include hydrofoil impellers, high-solidity pitch-blade impellers, high-solidity hydrofoil impellers, Rushton impellers, pitched-blade impellers, gentle marine-blade impellers, and the like. When containing two or more impellers, the impellers can be spaced apart along the rotating shaft 14.

As shown in FIG. 1B, the bioreactor 10 also includes a plurality of ports. The ports can allow supply lines and feed lines into and out of the bioreactor 10 for adding and removing fluids and other materials. In addition, the one or more ports may be for connecting to one or more probes for monitoring conditions within the bioreactor 10. In addition, the bioreactor 10 and be placed in association with a load cell for measuring the mass of the culture within the bioreactor.

In the embodiment illustrated in FIG. 1B, the bioreactor 10 includes a bottom port 26 connected to an effluent 28 for withdrawing materials from the bioreactor continuously or periodically, such as, in one aspect, to function as a perfusion bioreactor. Thus, in one aspect, the bottom port may include a system of screens or filters in order to maintain the cells in the bioreactor while removing waste and spent matrix materials. In addition, the bioreactor 10 includes a plurality of top ports, such as ports 30, 32, and 34. Port 30 is in fluid communication with a first fluid feed 36, port 32 is in fluid communication with a second feed 38 and port 34 is in fluid communication with a third feed 40. The feeds 36, 38 and 40 are for feeding various different materials to the bioreactor 10, such as a nutrient media.

In addition to ports on the top and bottom of the bioreactor 10, the bioreactor can include ports located along the sidewall. For instance, the bioreactor 10 shown in FIG. 1B includes ports 44 and 46.

Ports 44 and 46 are in communication with a monitoring and control system that can maintain optimum concentrations of one or more parameters in the bioreactor 10 for propagating cell cultures or otherwise producing a bioproduct. In the embodiment illustrated, for example, port 44 is associated with a pH sensor 52, while port 46 is associated with a dissolved oxygen sensor 54. The pH sensor 52 and the dissolved oxygen sensor 54 are in communication with a controller 60. The system of the present disclosure can be configured to allow for the determination and the measurements of various parameters within a cell culture contained within the bioreactor 10. Some of the measurements can be made in line, such as pH and dissolved oxygen. Alternatively, however, measurements can be taken at line or off line. For example, in one embodiment, the bioreactor 10 can be in communication with a sampling station. Samples of the cell culture can be fed to the sampling station for taking various measurements. In still another embodiment, samples of the cell culture can be removed from the bioreactor and measured off line.

In accordance with the present disclosure, a plurality of parameters can be measured during growth of a cell culture within the bioreactor 10. In general, the parameter being controlled by the process and system of the present disclosure is measured in conjunction with one or more other parameters that can influence the concentration of the parameter being controlled. For example, in one embodiment, lactate concentration is measured within the cell culture in conjunction with at least one other lactate influencing parameter. The lactate influencing parameter can comprise, for instance, glutamate concentration, glucose concentration, an amino acid concentration such as asparagine concentration, or the like. In one embodiment, at line or off line analysis of the cell culture can be performed using any suitable instruments such as a NOVA Bioprofile 400 analyzer sold by Nova Biomedical. The above analyzer is capable of measuring lactate concentration in conjunction with one or more of the lactate influencing parameters.

In accordance with the present disclosure, the lactate concentration and the concentration of the one or more lactate influencing parameters in addition to various other conditions in the bioreactor can be fed to the controller 60. The controller includes a control model that, based on the inputted data, is capable of forecasting lactate concentration in the future as the cell culture continues to propagate. In one embodiment, for instance, the controller can provide an early warning system that produces a percent probability as to whether the lactate concentration at the end of the cell culture incubation period is within preset limits or if the cell culture will end in a lactate accumulating state. The controller 60 can also be configured to accurately predict lactate concentration into the future. For instance, in one embodiment, the controller can forecast a lactate concentration trajectory that predicts lactate concentration through the entire incubation period until the cell culture is harvested. In one embodiment, the controller can also be configured to suggest or automatically implement corrective actions in case lactate concentration is not within preset limits. For example, the controller can be configured to determine nutrient feed changes, or changes in other operating conditions that may be required to drive the lactate concentration to a desired value. In order to determine corrective actions, the controller may run multiple iterations for determining future lactate concentrations based on altering one or more conditions within the bioreactor until an optimized change in one or more conditions is selected.

The controller 60 may comprise one or more programmable devices or microprocessors. As shown in FIG. 1B, the controller 60 can be in communication with the one or more feeds 36, 38 and 40, with one or more effluents 28, and/or with one or more propellers 16/18. In addition, the controller 60 can be in communication with the pH sensor 52, the dissolved oxygen sensor 54, and the gas supply 48 that feeds gas to the sparger 20. The controller 60 can be configured to increase or decrease the flow of materials into and out of the bioreactor 10 based upon the lactate concentration and the concentration of one or more lactate influencing parameters. In this manner, the controller 60 can maintain lactate concentration within preset limits. The controller 60 can operate in an open loop control system or can operate in a closed loop control system, where adjustments to input and/or output devices are completely automated. In other embodiments, the controller 60 can suggest corrective actions in order to influence lactate concentration and the corrective actions can be done manually.

Regardless of the bioreactor and/or bioreactor selected, in one aspect, the cells harvested from the 2D seed train cell stack 106 may be inoculated into a bioreactor containing a nutrient media/medium, which may be the same medium as discussed above. In one aspect the nutrient medium may be pre-placed in the reactor in an amount such that the volume of the nutrient medium has a volume that is about 30% or less of the volume of the bioreactor, such as about 40% or less of the volume of the bioreactor, such as about 50% or less of the volume of the bioreactor, such as about 60% or less of the volume of the bioreactor, such as about 66% or less of the volume of the bioreactor, such as about 70% or less of the volume of the bioreactor, and, in one aspect, a volume of the nutrient medium may be placed in the bioreactor such that the nutrient medium has a volume of about 60% to about 70% of the volume of the bioreactor.

Nonetheless, in one aspect, microcarriers may also be present in the bioreactor in addition to the nutrient medium prior to inoculating the bioreactor. In one aspect, the microcarriers may be introduced into the bioreactor along with the nutrient medium, or may be added after the nutrient medium but prior to inoculation. In a further aspect, the above discussed volume of nutrient media may be present in the bioreactor, and the microcarriers may be added after the initial volume of nutrient media but may be incorporated as part of a second volume of nutrient media. In such an aspect, the second volume of nutrient media containing the microcarriers may be about 10% or less of the volume of the bioreactor, such as about 15% or less of the volume of the bioreactor, such as about 20% or less of the volume of the bioreactor, such as about 25% or less of the volume of the bioreactor, such as about 20% or less of the volume of the bioreactor, such as about 33% or less of the volume of the bioreactor, such as about 35% or less of the volume of the bioreactor, and, in one aspect, a volume of the nutrient medium may be placed in the bioreactor such that the nutrient medium has a volume of about 30% to about 40% of the volume of the bioreactor.

Regardless of the manner in which the microcarrier are introduced, in one aspect, microcarriers are added to the bioreactor to promote cell growth. For instance, cells can adhere to the surface of the microcarriers for further growth and propagation. In this manner, the microcarriers can provide greater surface area for cell culture growth within the reactor. In fact, some anchorage-dependent cells, such as certain animal cells, need to attach to a surface in order to grow and divide. In some systems, the microcarriers are suspended within a nutrient medium caused by general agitation prior to, during, and/or after introduction to the bioreactor, which optimizes and maximizes the growing conditions within the bioreactor system.

Microcarriers can be made from various different materials, including polymers. The microcarriers can have any suitable shape and, in some applications, comprise round beads. In one aspect, microcarriers can generally have a median particle size of from about 50 μm to about 350 μm, such as from about 75 μm to about 300 μm, such as from about 100 μm to about 250 μm, such as from about 125 μm to about 225 μm, or any ranges or values therebetween. Previously, it was believed that small microcarriers (e.g. about 90-150 μm) were necessary for optimal expansion. However, the present disclosure has unexpectedly found that larger microcarriers (e.g. greater than 125 μm) may be used in combination with the process described herein, and yield expansion results, as good as, or better than results obtained with small microcarriers. Therefore, in one aspect, the microcarriers have a median particle size of about 125 μm or greater, such as about 150 μm or greater, such as about 175 μm or greater, such as about 200 μm or greater, such as about 210 μm or greater, such as about 350 μm or less, such as about 325 μm or less, such as about 300 μm or less, such as about 275 μm or less, such as about 250 μm or less, or any ranges or values therebetween. This provides a further benefit as small microcarriers are difficult to obtain, often requiring specialized equipment, thus larger particle sizes allow greater flexibility in scale-up of end-to-end expansion platforms.

Furthermore, in one aspect, the microcarriers may also be coated with a nutrient medium prior to introduction to a bioreactor and/or suspension in a nutrient medium. Particularly, the present disclosure has found that iPSCs showed improved growth and expansion when used with coated microcarriers as compared to uncoated microcarriers. Thus, in one aspect, the microcarriers may be coated with a nutrient matrix as discussed, such as a nutrient matrix as discussed above. Furthermore, in one aspect, the microcarriers may be coated in the same medium in which they are (or will be) suspended, or alternatively, may be coated in a different medium than the nutrient medium in which they will be supported. In yet a further aspect, the medium may be largely the same for coating and the support medium, but the coating medium and/or support medium may have one or more different additives.

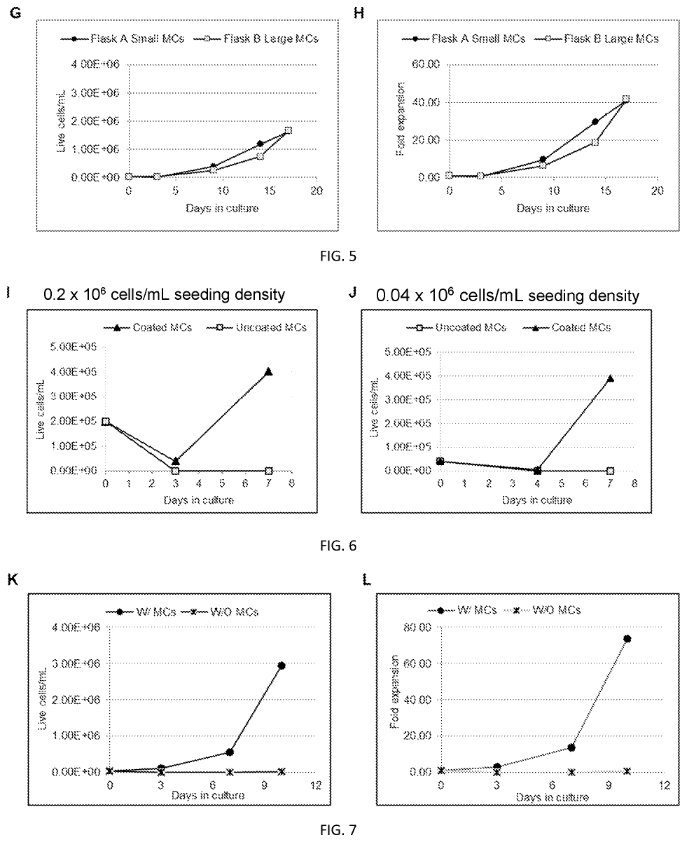

Notwithstanding the nutrient medium and microcarriers selected, the bioreactor may be inoculated with cells as discussed above at a seeding density of about 0.01×10⁶ cells/cm² to about 0.2×10⁶ cells/cm², such as about 0.02×10⁶ cells/cm² to about 0.15×10⁶ cells/cm², such as about 0.03×10⁶ cells/cm² to about 0.1×10⁶ cells/cm², such as about 0.04×10⁶ cells/cm² to about 0.07×10⁶ cells/cm². Particularly, as discussed above, it was previously believed that a high seeding density of 0.2×10⁶ cells/cm² was necessary for a 3 L or larger bioreactor to yield good expansion results. However, as will be discussed in greater detail below in regards to FIGS. 4 and 5, the present disclosure has found that small seeding densities (e.g. lower than 0.2×10⁶ cells/cm²) may be used in conjunction with the present disclosure, and yield excellent expansion results.

For instance, the present disclosure has found that low seeding densities may actually allow for higher fold expansion than higher seeding densities, such as a fold expansion of about 50 times or greater, such as about 60 times or greater, such as about 70 times or greater, such as about 80 times or greater, such as about 90 times or greater, such as about 100 times or greater, such as, in one aspect, about 50 times to about 120 times, such as about 60 times to about 100 times, such as about 70 times to about 95 times, such as about 80 times to about 90 times, or any ranges or values therebetween. Furthermore, the present disclosure has found that, unexpectedly, the expansion may take less time than an incubation that begins with a higher seeding density. For instance, the above expansions may take place in about 7 to about 18 days, such as about 8 to about 16 such as about 9 to about 14 days, and in one aspect, may reach a desired expansion (or seeding density) at a time less than a platform seeded with a high seeding density.

As discussed above, in one aspect, after the nutrient media, microcarriers, and inoculum have been introduced to the bioreactor, the contents of the bioreactor may be subjected to agitation. In one aspect, the bioreactor may be subjected to continuous gentle agitation at about 25 rpm to about 125 rpm, such as about 35 rpm to about 110 rpm, such as about 40 rpm to about 100 rpm, such as about 45 rpm to about 95 rpm, such as about 50 rpm to about 90 rpm, or any ranges or values therebetween. However, in a further aspect, the present disclosure has found that cell expansion may be further improved by a stepped agitation based upon cell density. For instance, in one aspect, the agitation may be increased every other day, such as every third day, such as every fourth day, such as every fifth day, by increasing the rpms by at least about 5 rpms, such as at least about 10 rpms, such as at least about 15 rpms, such as at least about 20 rpms, such as at least about 25 rpms, such as about 30 rpms or less.

Additionally or alternatively, the increase in rpms may be based upon a cell density measurement. For instance, in one aspect, the initial agitation speed may be set to about 25 rpm to about 75 rpm, such as about 35 rpm to about 65 rpm, such as about 40 rpm to about 60 rpm, such as about 45 rpm to about 55 rpm. Cell density measurements may be conducted, and when a cell density reaches about about 1×10⁵ cells/cm² to about 10×10⁵ cells/cm², such as about 3×10⁵ cells/cm² to about 8×10⁵ cells/cm², such as about 5×10⁵ cells/cm² to about 7×10⁵ cells/cm², an agitation speed may be increased by about 5 rpms, such as at least about 10 rpms, such as at least about 15 rpms, such as at least about 20 rpms, such as at least about 25 rpms, such as about 30 rpms or less.

Furthermore, in one aspect, after the agitation speed may be increased at least a second time. For instance, cell density measurements may be conducted again (or continuously conducted), and when a cell density reaches about about 4×10⁵ cells/cm² to about 5×10⁶ cells/cm², such as about 4.5×10⁵ cells/cm² to about 4×10⁶ cells/cm², such as about 5×10⁵ cells/cm² to about 3×10⁶ cells/cm², an agitation speed may be increased again by about 5 rpms, such as at least about 10 rpms, such as at least about 15 rpms, such as at least about 20 rpms, such as at least about 25 rpms, such as about 30 rpms or less.

In one aspect, the present disclosure has also found that discontinuous agitation on seeding day (first 24 hours of incubation) may further improve cell viability and expansion. Furthermore, the present disclosure has found that the discontinuous agitation may also be cascading agitation, such that earlier agitation is shorter, with increasing length of agitation and decreasing rest between agitation as time progresses. For instance, please refer to the characterization curve in 5.20.3 below. In such an aspect, the discontinuous and/or discontinuous cascading agitation may take place for the first 24 hours or less after inoculation, such as about 20 hours or less, such as about 18 hours or less, such as about 14 hours or less, such as about 10 hours or less after inoculation.

Nonetheless, when a desired cell density is reached, the expanded cells may be harvested 110. Namely, in one aspect, the expanded cells may be passaged and separated from the microcarriers by a non-enzymatic passaging solution, which may be the same passaging solution as discussed above, or may be a second passaging solution. Additionally, it should be understood that the passaging solution as discussed above may also include a kinase inhibitor. Furthermore, in one aspect, the passaging solution may also be combined with a nutrient media for passaging the cells from the bioreactor to the harvesting bag 110. Regardless, in one aspect, the cells are separated from the microcarriers by using an appropriate passaging solution, and are passed through a mesh having a mesh size selected to catch the microcarriers while allowing the cells to proceed through tubing to the harvest bag. For instance, in one aspect, the harvest bag assembly may have a mesh having a mesh size of about 10 μm to about 100 μm, such as about 25 μm to about 75 μm, such as about 50 μm to about 70 μm, or any ranges or values therebetween.

After the expanded cells have been harvested, the cells may be concentrated 112, such as by centrifugation. In one aspect, a flow rate through the centrifuge is selected based upon the formation of a fluidized bed. For instance, a flow rate may be optimized in order to minimize the time needed to establish the fluidized bed, maximize cell recover, and maintain cell viability and proliferation. Particularly, the present disclosure has found that cell retention and viability may be increased by establishing a fluidized bed in a short amount of time (such as about 15 minutes or less, such as from about 9 to about 13 minutes, such as about 10 minutes to about 12 minutes, in one aspect), and by minimizing a percentage of cells escaping the fluidized bed. For instance, in one aspect, the optimized fluidized bed may retain about 70% or greater of the cells, such as about 80% or greater, such as about 90% or greater of the cells entering the fluidized bed.

Nonetheless, after concentration, the cells may be filled and 114 and preserved by cryopreservation as may be known in the art.

While passaging has been discussed during several steps of FIG. 1, it should be understood and acknowledged that, unexpectedly, no passaging is needed during the incubation time, namely that greater than ten-fold expansion can be achieved in a continuous suspension culture according to the present disclosure.

Furthermore, as mentioned above, while 2D seed train steps 104 and 106 have been discussed in regards to FIG. 1, it should be understood that the present disclosure has also found that 3D seed train cells formed according to the present disclosure may be used to directly inoculate the bioreactor 108. Thus, in one aspect, steps 104 and 106 may be eliminated, and instead, cryopreserved cells 116 may be used as cryopreserved cells 102, and placed directly into bioreactor step 108. This finding is important for continued scale up of the platform, as larger end-to-end platforms (such as larger volume platforms) require larger and larger numbers of cells for inoculation. Therefore, a process for producing 3D seed train cells enables continued growth of scale-up, as the number of cells produced via a 3 L end-to-end platform far exceed 2D seed train production over the same time period.

For instance, the present disclosure has found that an end-to-end platform using a process according to the present disclosure may yield about 1 million cells/mL to about 5 million cells/mL, such as about 1.5 million cells/mL to about 4.5 million cells/mL, such as about 2 million cells/mL to about 4 million cells/mL. Additionally, the present disclosure has found that the process and platform according to the present disclosure may exhibit a cell retention after concentration of about 70% or greater, such as about 75% or greater, such as about 80% or greater, such as about 85% or greater, such as about 90% or greater. This finding also provides a further benefit, as 2D seed train provides high risk of contamination in addition to being time consuming and difficult. Therefore, direct inoculation with 3D seed train may also improve a lowered risk of contamination.

Additionally, the present disclosure has found that the end-to-end platform may be configured to be a closed system, and, in one aspect, may use single use vessels and tubing. Therefore, the end-to-end platform may further lower risk of contamination.

Furthermore, while the discussion has thus far focused on human pluripotent stem cells, it should be understood that other appropriate cells may be selected to undergo expansion according to the process and platform discussed herein. Additionally, as may be understood by one having skill in the art, the iPSCs discussed herein may be used as intermediates for any number of cells, as, will be discussed in greater detail below, the iPSCs expanded herein show excellent viability and differentiation.

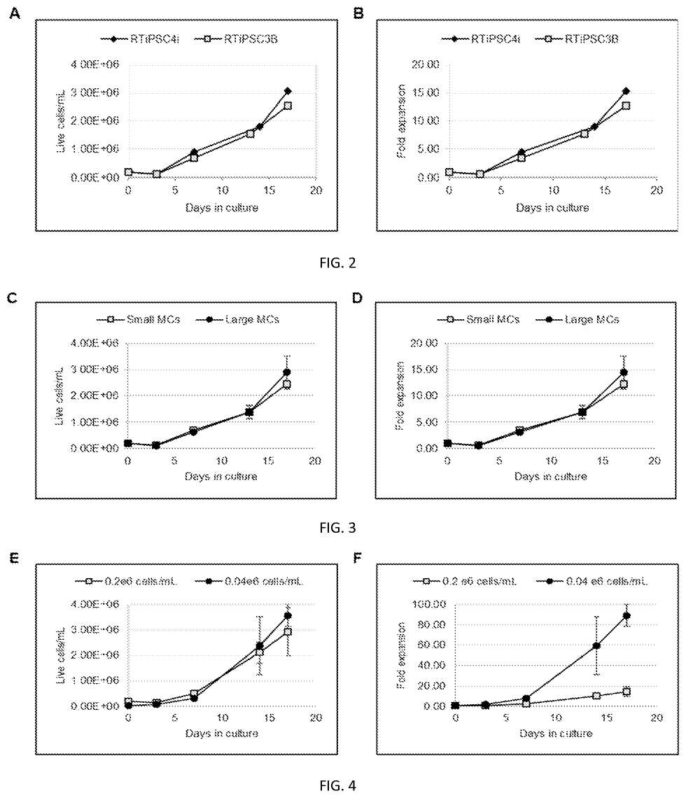

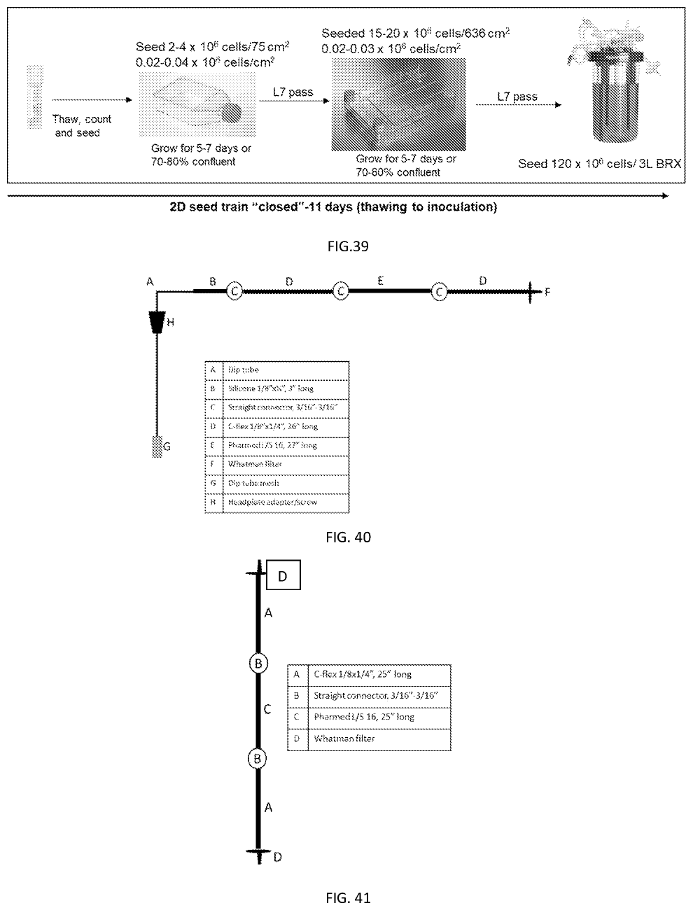

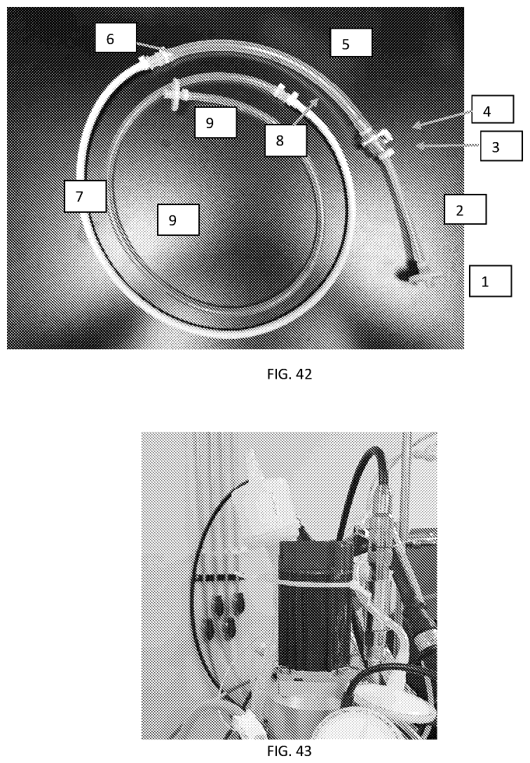

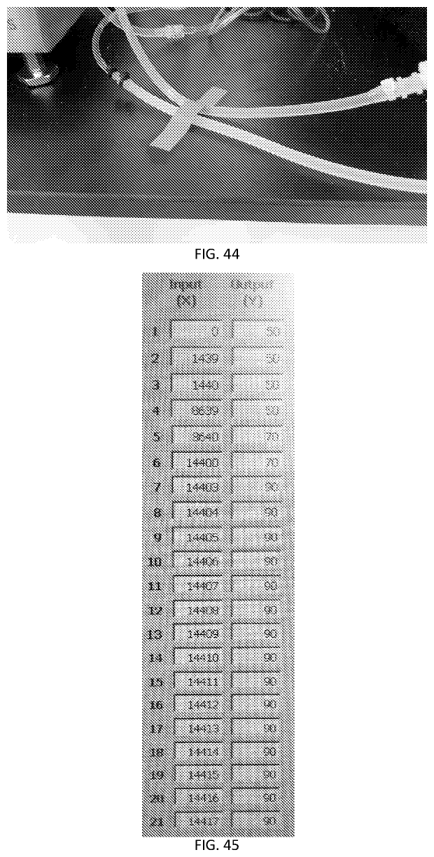

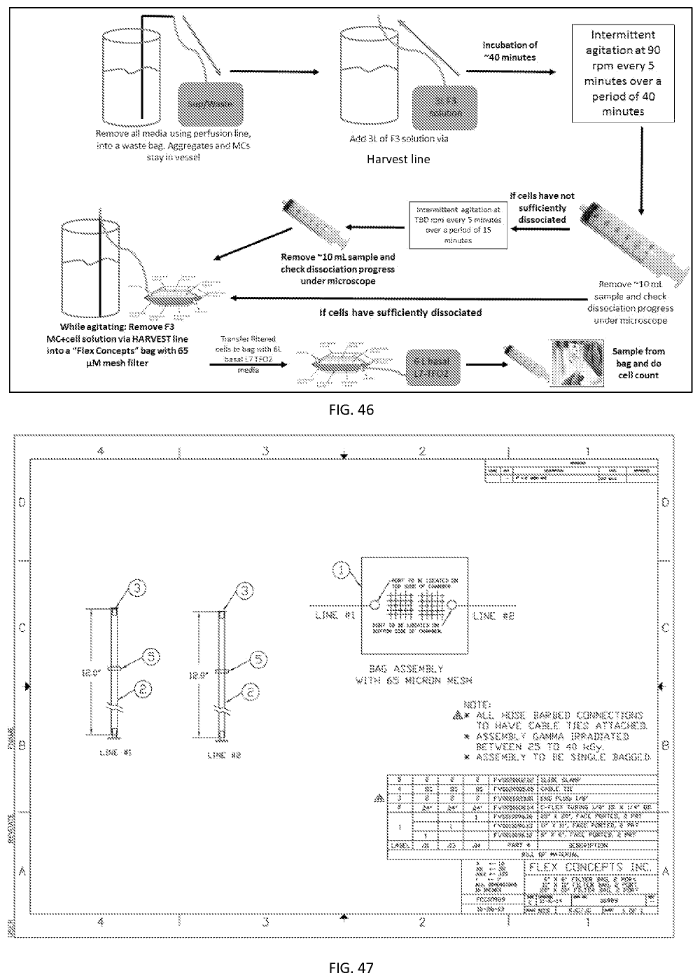

Nonetheless, further aspects of the present disclosure will now be discussed in regards to FIGS. 2-47 and the exemplary standard operating procedure.

Unless otherwise noted, FIGS. 2-47, and the examples underlying these figures, utilized the following materials and methods:

L7™ hPSC Culture System

Lonza L7™ culture system was developed for culturing hESCs and hiPSCs in feeder-free environment, and allows feeding on an every-other-day media change schedule. The culture system is comprised of recombinant, xeno-free and defined L7™ hPSC Matrix (Lonza, FP-5020) to enable cell attachment, xeno-free L7™ hPSC basal medium, xeno-free L7™ hPSC medium supplement, and non-enzymatic passaging solutions: L7™ hPSC passaging solution (yields cell clumps, Lonza, FP-5013) or F3 hPSC passaging solution (yields single cells). The L7™ hPSC basal medium used in the work described herein was modified by replacing native, animal-based components with respective recombinant components. It is referred to as L7™ TFO2 hPSC basal medium herein.

Human iPSC Lines

The human LiPSC18R iPSC line was generated from CD34+ cord blood cells as previously described (see, e.g. Baghbaderani, B. A. et al. Detailed Characterization of Human Induced Pluripotent Stem Cells Manufactured for Therapeutic Applications. Stem Cell Rev. Reports (2016) doi:10.1007/s12015-016-9662-8). RTiPSC3B and RTiPSC4i were generated from human peripheral blood mononuclear cells (PBMNCs, Lonza, CC-2702) of two different donors. Cryopreserved PBMNCs were thawed and cultured for six days in a priming medium comprised of an animal-free HPGM™ (Hematopoietic Progenitor Growth Medium, equivalent to Lonza, PT-3926, where native components were replaced with the respective recombinant ones), supplemented with 100 ng/mL recombinant human (rh) stem cell factor (SCF) (PeproTech, AF-300-07), 40 ng/mL insulin-like growth factor (IGF)-1 (PeproTech, AF-100-11), 10 ng/mL Interleukin (IL)-3 (PeproTech, AF-200-03), 1 μM Dexamethasone (Sigma, D1756), 100 μg/mL holo-transferrin (R&D Systems, 2914-HT) and 200 μM 1-Thioglycerol (Sigma, M6145). The PBMNCs were seeded in 6-well plates (Corning, 353046) at a density of 2-4×10⁶ cells/mL. On day 3, cells were collected, counted and seeded in a fresh priming medium at a density of 0.5-1×10⁶ cells/mL. On day 6, cells were collected, and cellular reprogramming was performed.

To reprogram the cells, 1×10⁶ PBMNCs were nucleofected with the episomal plasmids pCE-hOCT3/4, pCE-hSK, pCE-hUL, pCE-mP53DD and pCXB-EBNA-1 [37]. Nucleofection was performed using the 4D-Nucleofector™ System and P3 solution Kit (Lonza, V4XP-3012). After nucleofection, the cells were plated onto 6-well plates pre-coated with L7™ hPSC Matrix, in priming medium containing 0.5 mM Sodium Butyrate (Stemgent, 04-0005) to enhance reprogramming efficiency. Plates were placed in a 37° C. humidified incubator (5% CO2 and 3% O2). Two days post plating, L7™ hPSC medium was added to the wells without removing the priming media (1:1 ratio). On day 4, the media was aspirated and fresh L7™ hPSC medium, containing 0.5 mM Sodium Butyrate was added. Media change was performed in an every-other-day manner, and cells were incubated in a 37° C. humidified incubator (5% CO2 and 3% O2) until hiPSC colonies formed and were isolated for further expansion and characterization. These iPSC lines were characterized, showing normal karyotype, expression of key hPSC-associated markers, and demonstrating the potential to differentiate to cells of the three germ layers.

Culturing hiPSCs in 2D

Human iPSCs were cultured in 2D for the purpose of expanding cells for inoculation in a suspension vessel (spinner flask or stirred tank bioreactor) and characterization following expansion in suspension. hiPSCs were cultured in Lonza L7™ hPSC culture system using the animal-free L7™ TFO2 media and xeno-free L7™ hPSC media supplement. Cells maintained in culture were passaged and harvested either as cell clumps by L7™ hPSC Passaging solution (Lonza, FP-5013) or as single cells with F3 passaging solution to yield single cells and supplemented with 10 μM Y27632 (Stemgent, 04-0012) at plating.

For the culture of the 2D seed train prior to inoculation in a 3 L bioreactor, human iPSCs were thawed and plated onto L7™ hPSC matrix-coated T-75 culture flask and maintained in L7™ TFO2 media at a cell density of 0.02-0.04×10⁶ live cells/cm2. When the 2D culture reached 70-80% confluence, iPSC colonies were dissociated into cell clumps using the L7™ hPSC Passaging Solution and plated onto a 1-layer CellStack (Corning, 05-539-094) at a cell density range of 0.02-0.03×10⁶ live cells/cm². When the 2D culture reached 70-80% confluence, iPSC colonies were dissociated into cell clumps using the L7™ hPSC Passaging solution and 62-120×10⁶ live cells were inoculated in a 3 L bioreactor. The NucleoCounter NC-200 (Chemometec, Denmark) was used to assess the number and viability of the bioreactor cell inoculum.

Microcarrier Coating

Plastic microcarriers (MCs) (SoloHill brand polystyrene 90-150 μm, Pall Corporation, P-215-020 or 125-212 μm, Pall Corporation, P-221-020) were suspended in Dulbecco's Phosphate-Buffered Saline with calcium and magnesium (DPBS+/+, Lonza, 17-513F) and coated with L7™ hPSC Matrix. MCs and L7™ hPSC Matrix were incubated for 2 hours at 37° C. The DPBS+/+ solution was then aspirated, and the MCs were re-suspended in L7™ TFO2 hPSC basal medium and allowed to incubate overnight at room temperature with agitation. For expansion in 125 mL spinner flasks, 600 mg of MCs were incubated with 540 μg of L7™ hPSC Matrix. For expansion in a 3 L stirred tank bioreactor, 20 g of MCs were incubated with 18 mg of L7™ hPSC Matrix.

Culturing hiPSCs in a Spinner Flask

Human iPSCs were harvested from 2D as cell clumps or thawed directly as single cells into a 125 mL spinner flask (Corning, 3152) containing 100 mL L7™ media and L7™ hPSC Matrix-coated microcarriers (MCs). hiPSCs cultured in 2D were passaged with either L7™ hPSC Passaging solution to generate cell clumps or F3 passaging solution to dissociate colonies to single cells. The spinner flask was inoculated with either 0.04×10⁶ cells/mL of cell clumps or cryopreserved single cells. If single cells were inoculated, media was supplemented with 10 μM Y27632 (Stemgent, 04-0012). Spinner flask cultures were incubated in a 37° C. humidified incubator containing 5% CO2 overnight. After 24 hours, the spinner flask was placed on a magnetic stirring plate with an agitation speed of 25 RPM. The agitation speed was increased as needed to ensure hiPSC-MCs remain in suspension. The maximum agitation speed applied to support cell densities >2×10⁶ cells/mL was 90 RPM. Media was changed in an every-other-day manner using L7™ TFO2 media with xeno-free L7™ hPSC media supplement. To determine growth of hiPSCs in culture, 5 mL samples were obtained and hiPSCs were dissociated from the microcarriers using the F3 hPSC Passaging solution to generate single cells. The NucleoCounter NC-200 (Chemometec, Denmark) was used to determine cell number and viability.

hPSC Expansion in Stirred Tank Bioreactors

The BioBlu single-use bioreactor vessel was set up according to manufacturer's instructions (Eppendorf, 1386000300). Briefly, the 3 L vessel was equipped with probes required for online monitoring (Mettler Toledo) of key parameters including percentage of dissolved oxygen (DO), pH and temperature. The bioreactor was controlled using a G3 Lab Universal controller (Thermo Fisher Scientific). Prior to inoculation, L7™ hPSC Matrix-coated plastic microcarriers were introduced and the vessel was calibrated as previously described with L7™ TFO2 medium supplemented with L7™ hPSC medium supplement. The 3 L vessel was inoculated at day 0 with either 62-120×10⁶ 2D cultured cells in cell clumps (0.02-0.04×10⁶ cells/mL) or 204×10⁶ cryopreserved single cells at 37° C. with initial agitation rate set to 50 RPM. On day 1, perfusion with fresh L7™ TFO2 supplemented medium was initiated at a rate of one Vessel Volume per Day (VVD). Perfusion was performed using a proprietary designed microcarrier retention filter. To monitor the changes in key metabolites, 5 mL samples were taken from the bioreactor at various time points along the run. Offline monitoring to determine changes in parameters such as pH and key nutrients was performed using the BioProfile FLEX Analyzer (Nova Biomedical). To determine cell growth and fold expansion, 15 mL samples were taken in duplicates at various time points along the run and hiPSCs were dissociated from the microcarriers using the F3 hPSC passaging solution to generate single cells. The Nucleocounter NC-200 (Chemometec, Denmark) was used to measure the cell number and viability.

Harvest of hiPSCs from Stirred Tank Bioreactor

Human iPSCs in the 3 L bioreactor were collected upon reaching approximately >2×10⁶ cells/mL. With continuous agitation, the medium was first removed from the vessel. Warm F3 passaging solution was introduced to the vessel with continuous agitation. To verify detachment of hiPSCs from microcarriers, a 5 mL sample was obtained after 25-30 minutes of incubation with F3 passaging solution. The solution containing single cell hiPSCs and microcarrier was then transferred through a 30-65 μM pore sized filtration bag (Flex Concepts, FCC03475.01). The single cells were ultimately transferred into a 3 L bag containing L7™ TFO2 medium supplemented with L7™ hPSC medium supplement. Harvested cells were subjected to various assays including performance evaluation, characterization, downstream processing, and cryopreservation. In bioreactor runs where the intent was to concentrate and cryopreserve the cells for future use, 10 μM Y27632 (Stemgent, 04-0012) was added to the L7™ TFO2 media after treatment with F3 passaging solution.

Downstream Processing: Flow Rate Optimization for Fluidized Bed Formation

A kSep (Sartorius) was fitted with a 400.50 rotor, which functions as a 1/3.5 scale-down model for the kSep400. The associated 400.50 single-use kits (chamber set and valve set) were then installed. A 3 L PSC suspension was harvested from a bioreactor and connected as the feed source. A solution of PlasmaLyte-A (Baxter) and (0.25%) human serum albumin (Octapharma) was used to prime the system and wash the cells. A static centrifugation speed of 782 g was used. To optimize the formation of the fluidized bed, 3 flow rates (25, 30, 35 m L/min) were tested in increasing order. Prior to each run, the feed source was sampled in triplicate to determine cell density going into the kSep. For the entirety of the concentration process, 5 mL samples were drawn from the stream exiting the kSep chamber and tested using the NucleoCounter NC-200 (Chemometec, Denmark) to monitor the amount of cells escaping the fluidized bed. After 1 L of cell suspension was processed, the kSep was stopped, the chamber was emptied, and the concentrated cells were collected. The kSep was reset, the tubing and chambers were purged, and the process was repeated until all flow rates had been tested and the feed source was depleted.

Downstream Processing: hPSC Concentration Post Full Harvest

A bag containing the filtered PSC suspension harvested from the bioreactor was sampled in triplicate, and the viabilities and cell densities were then determined using a NucleoCounter NC-200. The average viable cell density (VCD) was used to calculate the concentrated volume that would be harvested by the kSep using Equation 1.

$$\\ \begin{matrix}Concentrated Vol = \frac{( Feed VCD ) * ( Feed Vol )}{Target Concentrated VCD} & Equation I\end{matrix}$$

The kSep400 (Sartorius) was equipped with its respective single-use kits (chamber set and valve set). A 10 L bag of DPBS (−/−) (Lonza) was used to prime the system (no wash step was performed). The feed bag was then welded onto the kSep valve set. The process recipe primed the system, ramped the centrifuge to 1000 g, then pumped cell suspension into 1 chamber at a rate of 120 mL/min (3.5× the value determined in the optimization experiment, rounded down). These settings were maintained until the entirety of the feed was processed by the kSep. Periodically throughout the process, 5 mL samples were drawn from the stream exiting the kSep chamber and were tested using the NucleoCounter NC-200 to monitor the amount of cells escaping the fluidized bed. After the feed bag emptied, the concentrated cells were harvested. The volume of the concentrate was verified, and samples were taken to determine viability and cell density. The remaining concentrate was cryopreserved.

Cryopreservation

Human iPSCs were suspended in cryopreservation solution (CS10, Biolife Solutions Inc, 210102) containing 10 μM of Y-27632 (Stemgent, 04-0012). Cryovials were cryopreserved by Cryomed™ Controlled-rated Freezer (Thermo Fisher Scientific, Model 7456) and subsequently stored in liquid nitrogen until use.

Immunofluorescence Staining

Cells cultured in 2D were fixed with 4% paraformaldehyde (Santa Cruz, SC 281692) blocked with blocking solution comprised of 10% donkey serum and 0.1% Triton X-100 in PBS−/−. The cells were incubated with primary antibodies followed by secondary antibody incubation and DAPI staining. Immunofluorescence was observed using Olympus IX73 microscope. The following primary antibodies were used to detect hPSC-associated markers: OCT4/POU5F1 (Abcam, ab19857), NANOG (R&D systems, AF1997), TRA-1-81 (Stemgent, 09-0011), TRA-1-60 (Millipore, MAB4360), SSEA-4 (Millipore, MAB4304). The following primary antibodies were used to detect expression of germ-layer specific markers: SOX17 (R&D systems, AF1924), FOXA2 (Abcam, Ab108422), NESTIN (R&D systems, MAB1259), PAX6 (Biolegend, #901301), α-actinin (Sigma, A7811) and SMA (Millipore, CBL171).

Flow Cytometry

Quantitative detection of hPSC-associated markers was performed using flow cytometry as previously described (see, e.g. Shafa, M., Panchalingam, K. M., Walsh, T., Richardson, T. & Baghbaderani, B. A. Computational fluid dynamics modeling, a novel, and effective approach for developing scalable cell therapy manufacturing processes. Biotechnol. Bioeng. (2019) doi:10.1002/bit.27159, Baghbaderani, B. A. et al. CGMP-manufactured human induced pluripotent stem cells are available for pre-clinical and clinical applications. Stem Cell Reports (2015) doi:10.1016/j.stemcr.2015.08.015, Shafa, M., Yang, F., Fellner, T., Rao, M. S. & Baghbaderani, B. A. Human-induced pluripotent stem cells manufactured using a current good manufacturing practice-compliant process differentiate into clinically relevant cells from three germ layers. Front. Med. (2018) doi:10.3389/fmed.2018.00069). Briefly, single cells were live-stained for the cell surface markers: TRA-1-81 (BD Biosciences, #560161), TRA-1-60 (BD Biosciences, #560884) and SSEA-4 (BD Biosciences, #560126). Cells were also fixed, permeabilized and stained for OCT4/POU5F1 (Cell Signaling, #5177S). The samples were processed using either FACSCanto™ II (Becton Dickinson) or the FACSCelesta™ (Becton Dickinson), and data was acquired using the BD FACSDiva Software followed by analysis using FlowJo v10 software (FlowJo).

Alkaline Phosphatase Staining

Alkaline phosphatase staining was performed using StemAb Alkaline Phosphatase Staining Kit II (Stemgent, 00-0055), following manufacturer instructions

Karyotyping

Live cells were plated onto a T-25 flask, pre-coated with L7™ hPSC Matrix and maintained in L7™ TFO2 hPSC medium. Karyotype analysis (G-banding) was performed at LabCorp (Santa Fe, New Mexico).

Embryoid Body Formation

Embryoid body (EB) formation was performed by plating single cells in AggreWell800 (Stem Cell Technologies, 34811) in medium containing Knockout DMEM F-12 (Gibco, 12660-012), 20% Knockout Serum (Gibco, 10828-028), non-essential amino acids-1× (Gibco, 11140-050) and Glutamax-1× (Gibco, 35050-061). Medium was changed 48 hours later and thereafter till day 7 in an every-other-day mode. On day 7, EB spheres were collected and plated onto plates coated with 0.1% Gelatin (Millipore, ES-006-B) and medium containing DMEM (Gibco 11965-092), 20% FBS (Gibco, SH30071), non-essential amino acids-1× (Gibco, 11140-050) and Glutamax-1× (Gibco, 35050-061). Medium was changed every other day for 7 days. On day 7 post plating, EBs were fixed with 4% paraformaldehyde (Santa Cruz, SC-281692), and stained for detection of cells of the three germ layers with antibodies for the following antigens: SOX17 (R&D Systems, AF1924) for endoderm, PAX6 (BioLegend, PRB-278P) for ectoderm, and SMA (Millipore, CBL171) for mesoderm.

Definitive Endoderm Differentiation

Human iPSCs were differentiated into definitive endoderm (DE) as previously described [39]. Briefly, 0.25×10⁶ single cells were seeded onto L7™ hPSC Matrix-coated 24-well plates on day 0 with L7™ TFO2 media containing L7™ hPSC medium supplement and 10 μM Y27632 (Stemgent, 04-0012). On day 1, the STEMdiff™ Definitive Endoderm Kit (Stem Cell Technologies, 05110) was used to induce DE differentiation according to manufacturer's protocol. The cells were washed, fixed on day 5 and stained for DE-specific markers SOX17 (R&D systems, AF1924) and FOXA2 (Abcam, Ab108422).

Neural Stem Cell Differentiation

Human iPSCs were differentiated into neural stem cells (NSCs) as previously described [39]. In brief, 0.25×10⁶ single cells were seeded onto L7™ hPSC Matrix-coated 6-well plates on day 0 with L7™ TFO2 media containing L7™ hPSC medium supplement and 10 μM Y27632 (Stemgent, 04-0012). On day 1, the culture medium was changed with Neural Induction Medium (NIM) composed of B-27 Plus Neuronal Culture System (Gibco, A3653401) with 1× Glutamax (Gibco, 35050-061), 4 μM CHIR99021 (Stemgent, 04-0004-02), 3 μM SB431542 (Stemgent 04-0010-10) and 10 ng/mL hLIF (Peprotech, 300-00-250). NIM was changed in an every-other-day manner. When the cells reach 95-100% confluence, the cells were passaged as single cells using the F3 passaging solution. 1×10⁶ and 0.25×10⁶ cells were seeded onto 6- and 24-well plates, respectively (NSC-P1). NIM was replenished the following day and every-other-day till cell were fixed and stained for neural progenitor markers, NESTIN (R&D systems, MAB1259) and PAX6 (Biolegend, 901301). The cell culture plates used for NSC culture were pre-coated by incubating with 20 μg/mL Poly-L-ornithine (Sigma P4957) in sterile cell culture grade water (Lonza 17-524F) for 2 hours at 37° C. The plates were then washed with calcium and magnesium free DPBS (DPBS−/−) (Lonza, 17-512F), followed by a 1-hour incubation at 37° C. with 15 μg/mL Laminin (Sigma, 11243217001) re-suspended in DMEM/F12 (Thermo Fisher Scientific, 11330032) or PBS −/− (Lonza, 17-516F).

Cardiac Differentiation

Human iPSCs were differentiated into cardiomyocytes using the Gsk3 inhibitor and Wnt inhibitor (GiWi) protocol as previously described. (see, e.g. Lian, X. et al. Directed cardiomyocyte differentiation from human pluripotent stem cells by modulating Wnt/β-catenin signaling under fully defined conditions. Nat. Protoc. (2013) doi:10.1038/nprot.2012.150, Lian, X. et al. Robust cardiomyocyte differentiation from human pluripotent stem cells via temporal modulation of canonical Wnt signaling. Proc. Natl. Acad. Sci. U.S.A. (2012) doi:10.1073/pnas.1200250109; Zhang, J. et al. Functional cardiomyocytes derived from human induced pluripotent stem cells. Circ. Res. (2009) doi:10.1161/CIRCRESAHA.108.192237). Briefly, 1×10⁶ single cells/mL were seeded onto L7™ hPSC Matrix-coated 6-well plates in the presence of 10 μM Y27632 (Stemgent, 04-0012). Cells were maintained with L7™ TFO2 media containing L7™ hPSC medium supplement until confluence, during which time the cells were treated with 6-12 μM CHIR99021 (Tocris Bioscience, 4423) in RPMI/B27-insulin medium (day 0). After 24 hours, the medium was changed to fresh RPMI/B27-insulin (day 1). 5-7.5 μM IWP2 (Tocris Bioscience, 3533) was added on day 3, with fresh medium added on day 5. From day 7, cells were maintained in RPMI/B27 medium with medium changed in an every-other-day manner until spontaneous contraction was observed. Thereafter, iPSC-derived cardiomyocytes were dissociated as single cells using 10× TrypLE™ Select Enzyme (Thermo Fisher Scientific, 12563011) for 5-10 minutes at 37° C. Cells were plated on 24-well plates coated with 0.1% gelatin (Millipore, ES-006-B) in EB20 medium, which is comprised of DMEM/F12 (Thermo Fisher Scientific, 11330032), FBS (GE Healthcare, SH30071.01), MEM Non-Essential Amino Acids (Thermo Fisher Scientific, 11140050), GlutaMAX™ Supplement (Thermo Fisher Scientific, 35050061) and 2-Mercaptoethanol (Thermo Fisher Scientific, 21985023). The cells were fixed and stained for mesoderm-specific markers, α-ACTININ (Sigma, A7811) and smooth muscle actin (SMA) (Millipore, CBL171).

### FIGURES AND EXAMPLES

Referring first to FIG. 2, and as discussed above, it is shown that the process according to the present disclosure yields fold expansion of greater than 10 over a seventeen-day period. For instance, the hPSC culture system discussed herein, which, in this example, includes the L7™ TFO2 hPSC medium and matrix, supported the expansion of the hiPSCs tested in manually operated, open, spinner flasks and in automated, closed, stirred tank bioreactors. Particularly, RTiPSC4i and RTiPSC3B cells cultured in a xeno-free nutrient media supplemented with growth factors and cytokines optimized for long-term growth, were shown to exhibit excellent growth and expansion. Thus, in this example, L7™ TFO2 hPSC medium supported human iPSC generation from somatic cells such as fibroblast and PBMNCs. Furthermore, while the data is not shown, maintenance of various hESC and hiPSC lines in conventional 2D cell culture platforms, which utilize cell culture vessels coated with L7™ hPSC Matrix to support cell attachment, was also supported. To assess the ability of L7™ TFO2 hPSC media to support growth of hPSCs in suspension, 2D cultured RTiPSC3B and RTiPSC4i cells were harvested as cell clumps and inoculated in L7™ TFO2 hPSC medium at a cell density of 0.2×10⁶ cells/mL, with microcarriers 90-150 μM in diameter coated with L7™ hPSC Matrix. As indicated in FIGS. 2A and 2B, an initial decrease in cell number was observed during the first days in suspension followed by an increase in cell number, which reached >2×10⁶ cells/mL on day 17 (>10-fold expansion). The results indicate that L7™ TFO2 media supports MC-based expansion of hPSCs in suspension and that that 10-fold expansion could be achieved in a continuous suspension culture over 17 days without the need for cell passaging.

As shown in FIG. 3, the present disclosure has found that, contrary to prior teaching, larger diameter microcarriers (MCs) support expansion of HPSCs as well as, if not better, than small diameter microcarriers. Spinner flasks were inoculated with 0.2×10⁶ cells/mL of RTiPSC3B in the presence of 90-150 μM (small) or 125-212 μM (large) sized MCs coated with L7™ hPSC Matrix. Similar cell growth and expansion over 17 days was observed when using small and large sized MCs. In both conditions, cells reached >2×10⁶ cells/mL and >10-fold expansion on a similar day during culture. These results validate the use of larger sized MCs for cell expansion.

In FIGS. 4 and 5, the present disclosure has shown that, contrary to prior teaching, lower inoculation density does not compromise cell yield, and instead leads to higher fold expansion. To generate high cell numbers in suspension systems such as a 3 L bioreactor with an inoculum cell density of 0.2×10⁶ cells/mL, would require 600×10⁶ cells at inoculation. Spinner flasks were inoculated with RTiPSC4i cells at two different cell densities: 0.2×10⁶ cells/mL and 0.04×10⁶ cells/mL. The cells were cultured in suspension for 17 days and cell counts determined at various time points during the expansion period. FIG. 4 shows that comparable cell densities were achieved at both inoculum cell densities. The spinner flasks inoculated with a higher cell density resulted in 3×10⁶ cells/mL, while inoculating with a lower cell density resulted in 2.5×10⁶ cells/mL on day 17. Comparison of expansion fold results demonstrates that the higher density seeding resulted in ˜15-fold expansion by day 17, which is comparable to expansion fold described above in FIG. 2. However, the lower seeding density inoculum, which used five times fewer hiPSCs at inoculation, resulted in 90-fold expansion on day 17. This fold expansion is ˜6 times higher than that obtained using the higher seeding density.

To confirm these findings with a different hiPSC line, the present disclosure inoculated RTiPSC3B cells at a cell density of 0.04×10⁶ cells/mL with either small or large sized coated MCs. FIG. 5 demonstrated cell growth and fold expansion over 16 days. Cell yield reached >1.6×10⁶ cells/mL with expansion fold of >40 on either small or large sized MCs. Thus, the present disclosure has found that hiPSCs inoculated at lower cell densities are capable of achieving higher-fold expansion, easing the burden of large-scale expansion in 2D cell culture platforms prior to inoculation in 3D suspension culture.

Referring next to FIGS. 6 and 7, as discussed above the present disclosure has found that coating microcarriers in nutrient matrix may further enable hPSC attachment to the microcarriers. Cells were harvested from 2D cell culture using L7™ Passaging solution and seeded as cell clumps in spinner flasks at a density of 0.2×10⁶ cells/m L. Both flasks contained microcarriers, where in one flask the microcarriers were coated with L7™ hPSC Matrix and in the other, the microcarriers were not coated. While cell viability on day 0 (inoculation day) was determined to be 85%, a cell count performed on day 3 revealed a decrease in cell number. This observation matches previous results for reduced cell density in the first days in suspension (FIGS. 2 and 3). On day 7, however, cells that were seeded with L7™ hPSC Matrix-coated MCs showed both high viability (90%) and 2-fold expansion. Cells that were seeded with uncoated MCs failed to show growth and expansion. The results show that coating the MCs improves cell expansion. The experiment was repeated with a lower seeding cell density of 0.04×10⁶ cells/mL. LiPSC18R cells were inoculated in a spinner flask with either coated or uncoated MCs. Monitoring cell growth over 7 days showed that expansion was minimal in the spinner flask where cells were incubated with uncoated MCs, compared to 10-fold expansion on day 7 in the spinner flask where cells were incubated with coated MCs.

To rule out the possibility that the cells expanded without MCs, RTiPSC4i cells were inoculated in spinner flasks with and without large sized MCs coated with L7™ hPSC Matrix as shown in FIG. 7. In the flask that had MCs, cell reached 70-fold expansion in 10 days, while no expansion was detected in the flask that did not have MCs.

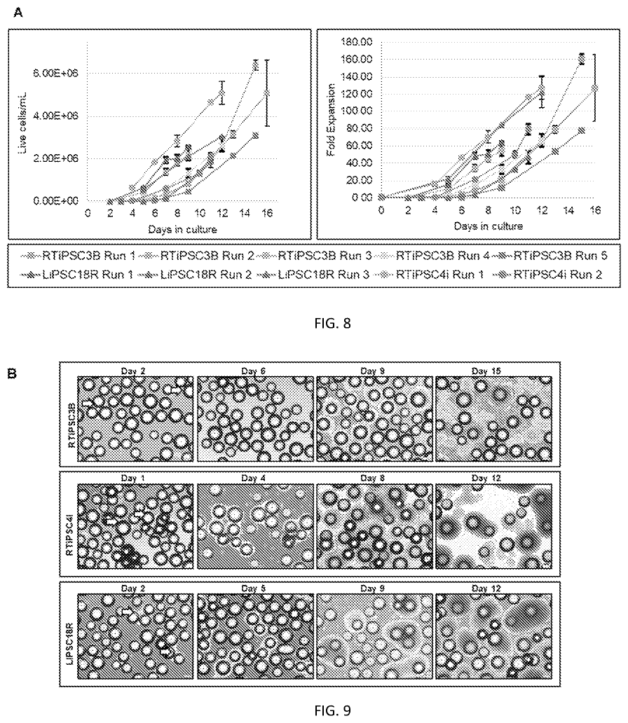

Referring next to FIGS. 8 and 9, the present disclosure has further found that the process described herein is scalable to larger bioreactors. The spinner flask experiments demonstrated that hiPSCs expanded in L7™ TFO2 media using L7™ hPSC Matrix-coated MCs resulted in high-fold expansion. To demonstrate scalability, a 3D expansion system in 1 L (data not shown) and 3 L stirred tank bioreactors were conducted. Ten 3 L bioreactor runs were performed using three different hiPSC lines harvested from 2D culture as cell clumps. Cells were inoculated at a cell density range of 0.02-0.04×10⁶ cells/mL with L7™ hPSC Matrix-coated MCs (125-212 μM) and maintained in L7™ TFO2 media, perfused at 1 vessel volume per day (VVD). Cell counts performed on single cells released from the MCs revealed an expansion of 5 to 20-fold in the first 5-9 days, and additional >10 fold expansion onward, resulting in >2×10⁶ cells/mL (FIG. 8). An average 93-fold expansion within 9-16 days of culture across all three hiPSC cell lines was achieved in these culture conditions. Notably, although RTiPSC4i Run 2 used only 0.027×10⁶ cells/mL at inoculation, approximately 80-fold expansion was still achieved in 11 days. This result supports the findings in the spinner flasks experiments, demonstrating that lower inoculum cell density does not compromise cell yield, supporting platform robustness. Cell viability along the various days of the bioreactors run was determined to be high (>85%, data not shown). Images of cell-MC samples taken from the bioreactor at various time points show cell expansion over time (FIG. 9).

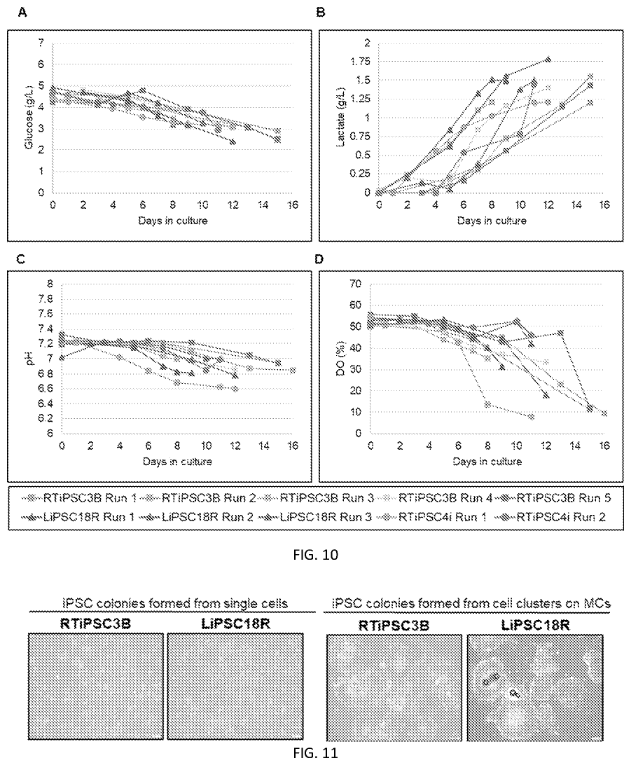

Furthermore, referring to FIG. 10, various metabolites were monitored during incubation. Surprisingly, the present disclosure has found that iPSC cells may have excellent growth and expansion with lower than previously believed levels of dissolved oxygen. Nonetheless, key nutrients, such as glucose, and metabolites, such as lactate, were monitored during run. FIG. 10 shows a decrease in glucose levels, corresponding to increased glucose consumption as the cells expand in culture. Glucose concentrations did not drop below 2.4 g/L, even when cell density in the bioreactor reached 3.05×10⁶ cells/mL (LiPSC18R Run 2). Conversely, lactate production increased with a maximum concentration of 1.79 g/L seen (LiPSC18R Run 2). For the other cells lines, lactate levels did not exceed 1.6 g/L even for high cell densities of 5.6×10⁶ cells/mL (RTiPSC3B) or 5.1×10⁶ cells/mL (RTiPSC4i). Additionally, pH and dissolved oxygen (DO) were closely monitored in real-time using the TruBio DV Software from Finesse Solutions. Similarly to the nutrient levels, pH levels were affected by cell expansion. While the set point was 7.2, as cells expanded, pH levels dropped to ˜6.8. Dissolved oxygen was set to 50% and maintained for the first few days of the run. As cell expanded, however, DO levels dropped and the Finesse controller could not maintain the set target. DO levels dropped to 30% for cell densities close to 2×10⁶ cells/mL. For higher cell densities of >4×10⁶ cells/m L, DO levels were below 10%.

Previous studies have demonstrated that 02 levels regulate hPSCs metabolic flux, but the expression of pluripotency and differentiation markers in hPSCs cultured in either 20% or 5% 02 was unaltered. Moreover as will be discussed in greater detail below, no differences in proliferation were observed, suggesting that low O₂ improves hPSC stemness. Others have also shown that 30% DO is the optimal condition to support hPSC expansion. In line with this study, we did not observe an adverse impact on the quality of cells harvested at cell densities of ˜2.5×10⁶ cells/mL where DO levels remained >30. Moreover, characterization of cells harvested from a 3 L bioreactor at a cell density of >5×10⁶ cells/mL, with corresponding DO levels of 10%, showed that the cells had normal karyotype, expressed hPSC-associated markers and were capable of differentiating into cells of the three germ layers. These findings suggest minimal impact of 02 levels on the quality of cells expanded in the end-to-end platform discussed herein.

Furthermore, referring to FIGS. 11-13, the present disclosure has found that the hPSCs formed according to the present disclosure exhibited excellent morphology and expression of hPSC-associated markers. Namely, cell harvest from the bioreactor was performed in a closed manner inside the bioreactor. Medium was pumped out, and F3 non-enzymatic passaging solution was pumped in to release the cells from the microcarriers. As a result of F3 passaging solution treatment, cells were released from the microcarriers as single cells. The resulting solution of single cells and microcarriers in F3 passaging solution were then transferred in a closed manner through a separation bag to separate the cells from the microcarriers. The cells passed through the filter bag directly into a collection bag containing L7™ TFO2 hPSC media. In order to assess the quality of expanded hiPSCs post-harvest from a bioreactor, the cells were characterized for morphology, hPSC-associated markers, validated their karyotype, and determined their pluripotency.

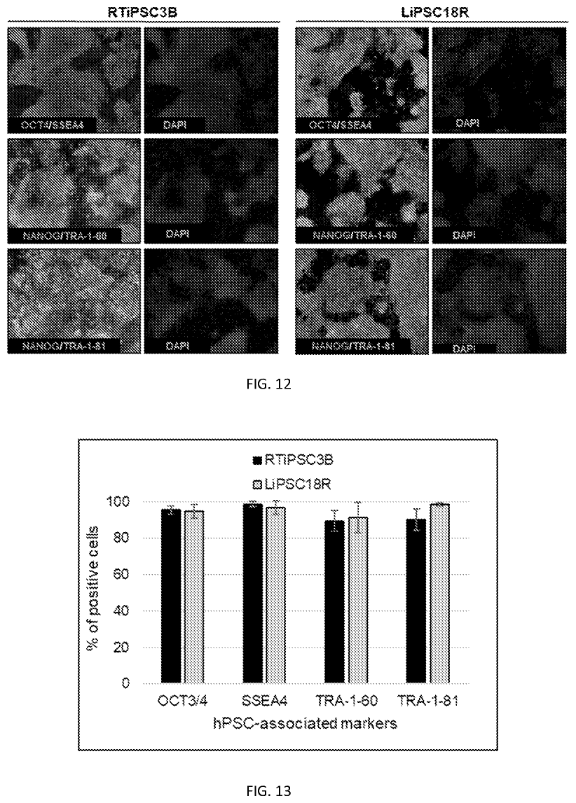

To assess morphology, hiPSCs on MCs or MC-released hiPSCs were plated onto 2D cell culture plates coated with L7™ hPSC Matrix. FIG. 11 shows that RTiPSC3B and LiPSC18R cells, cultured in 2D culture plates after harvest from a 3 L bioreactor, had a typical hPSC colony morphology, including defined edges and tightly packed cells with large nuclei and scant cytoplasm. Moreover, immunofluorescence staining of both cell lines shows qualitative expression of hPSC-associated markers (FIG. 12), while flow cytometry results further validated that >85% of the iPSCs expanded in the bioreactor expressed hPSC-associated markers post-harvest (FIG. 13). Karyotype analysis, reflected in Table 1 below, showed no genome abnormality in cells harvested from a bioreactor and cultured for one or more passages in 2D.

TABLE 1

| | Passage number at | | # of 20 passages | Cell processing | |
|---|---|---|---|---|---|
| Cell line | inoculation | Bioreactor size | post harvest | before karyotyping | Karyotype |
| RTiPSC3B | P [symbol] | 1 L | P1 | Expansion & harvest | Normal |
| RTiPSC3B | P [symbol] | 1 L | P1 | Expansion & harvest | Normal |
| [symbol] | P [symbol] | [symbol] L | P [symbol] | Expansion & harvest | Normal |
| [symbol] | P [symbol] | [symbol] L | P10 | Expansion, harvest, | Normal |
| | | | | culture in 20 | |
| RTiPSC3B | P [symbol] | 3 L | P [symbol] | Expansion & harvest | Normal |
| RTiPSC3B | P [symbol] | 3 L | P1 | Expansion & harvest | Normal |
| [symbol] | P [symbol] | 3 L | P [symbol] | Expansion & harvest | Normal |
| RTiPSC3B | P [symbol] | 3 L | P1 | Expansion & harvest | Normal |
| RTiPSC3B | P [symbol] | 3 L | P1 | Expansion & harvest | Normal |
| LiPSC18R | P [symbol] | 3 L | P1 | Expansion & harvest | Normal |
| LiPSC18R | P [symbol] | 3 L | P1 | Expansion, harvest | Normal |
| | | | | concentration | |
| LiPSC18R | P [symbol] | 3 L | P1 | Expansion, harvest | Normal |
| | | | | concentration | |

[symbol]

indicates data missing or illegible when filed

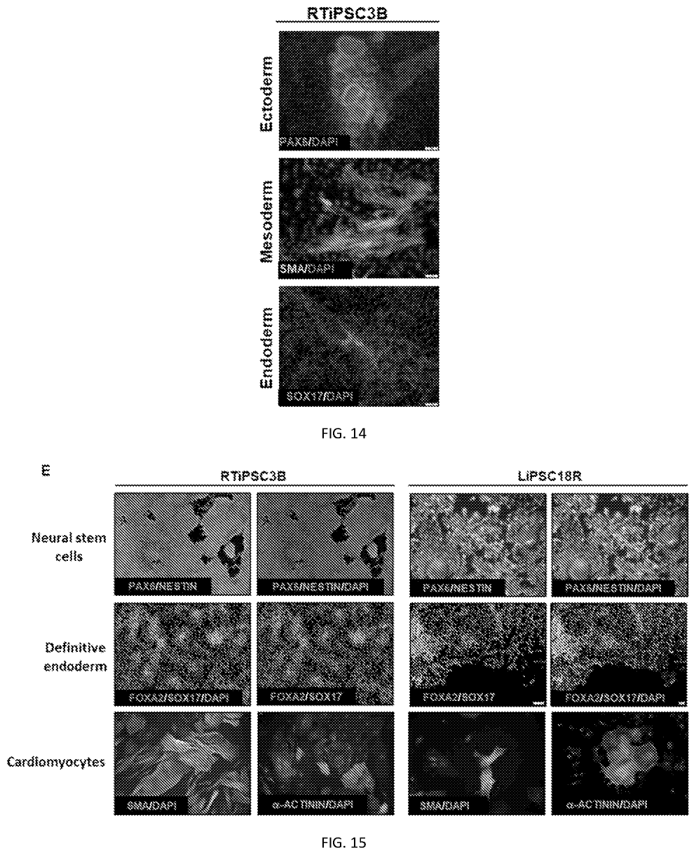

Furthermore, as shown in FIGS. 14 and 15, the potential of the expanded hiPSCs to differentiate into cells of the three germ layers was assessed either by embryoid body (EB) formation or by directed differentiation. FIG. 14 shows immunofluorescence staining images of plated EBs formed from RTiPSC3B cells expanded in a 3 L bioreactor. Positive detection of germ layer-specific markers demonstrates that cells expanded in a bioreactor retain their potential to give rise to cells of the three germ layers post-harvest. Directed differentiation of hiPSCs to definitive endoderm (DE, endoderm germ layer), neural stem cells (NSCs, ectoderm germ layer) and cardiomyocytes (CMs, mesoderm germ layer) was performed on RTiPSC3B and LiPSC18R cells after harvest from 3 L bioreactors. As shown in FIG. 15, immunostaining for germ-layer specific markers confirmed that the expanded cells retained pluripotency and capability to be directly differentiated to DE, NCSs and CMs. As described in Material and Methods section, immunostaining for CM-specific markers was performed after observing spontaneous contraction.

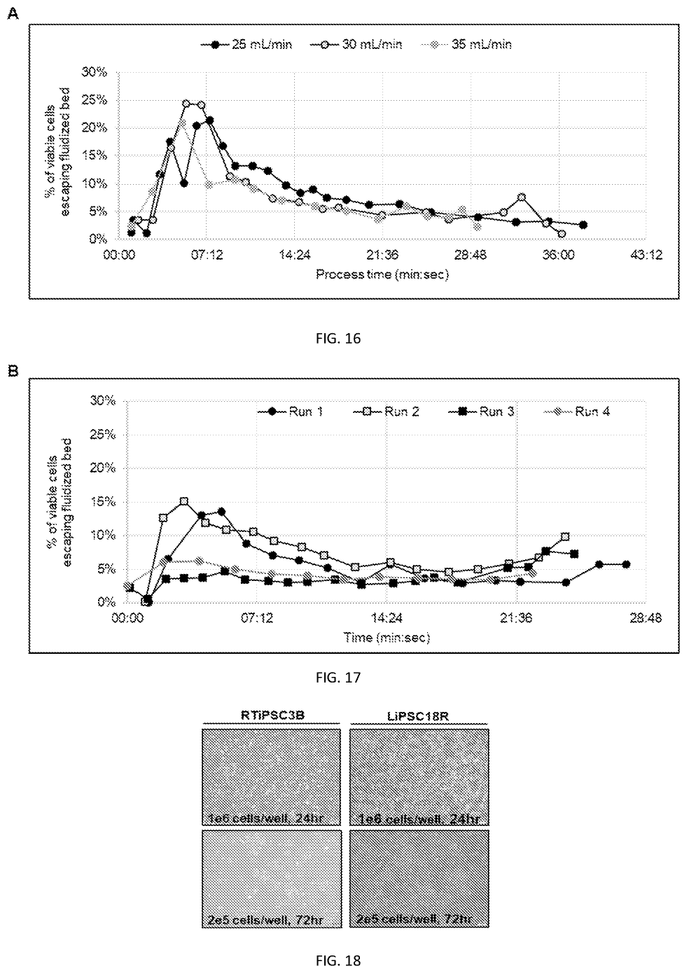

Referring next to FIGS. 16 and 17, as the manufacture of cell therapies advances towards larger scale cultures in bioreactors, more cells will be used for inoculation and more cells will need to be processed after harvesting. Common existing treatment of iPSCs after a bioreactor harvest is to have an operator wash out the culture media, concentrate the cells via benchtop centrifugation, and then re-suspend in cryoprotectant. Moving towards larger scale GMP production, this open step is a critical contamination risk to the product and ultimately to the patient who would receive it, and is difficult to scale. One solution is to make use of continuous centrifugation devices, for example a kSep400 continuous centrifugation system.

Optimization of the flow rate during fluidized bed formation was defined as (1) minimizing the time needed to establish the fluidized bed, (2) maximizing cell recovery, and (3) maintaining cell viability and proliferation capabilities. The establishment of the fluidized bed is achieved when the majority of cells coming into the kSep chamber are retained, and thus the percent of escaping cells reaches a minimum. Quantitatively, this can be defined as the escape percent dropping below 10%, meaning the fluidized bed captures 90% or more of the incoming cells.

The 30 mL/min and 35 mL/min established the fluidized bed in 10-11 minutes, and the 25 mL/min flow rate established the bed after 13-14 minutes (FIG. 6A). The cells from the 30 and 35 mL/min tests were taken for cell counts and culturing. The cell counts revealed that the cell viability was not negatively affected by the concentration process and that both protocols had recoveries 80% (see Table 2 below). The flow rate of 35 mL/min was selected for concentration of iPSCs in the kSep 400.50. This flow rate was scaled up to 120 mL/min when moving into the kSep400.

TABLE 2

| | | Before kSep | | | After kSep | | | | |
|---|---|---|---|---|---|---|---|---|---|
| | | Viable | Viable | | Viable | Viable | | | |
| Flow | Harvest | cells into | Cell | | cells | Cell | | Total | |
| Rate | volume | kSep | Density | Viability | harvested | Density | Viability | Cells | Recovery |
| (mL/min) | (mL) | (c) | (c/mL) | (%) | (c) | (c/mL) | (%) | Harvested | (%) |
| 30 | 25 | 3.20E+09 | 2.94E+06 | 87.60 | 2.55E+09 | 1.02E+08 | 90% | 2.84E+09 | 80% |
| 35 | 30 | 3.19E+09 | 3.11E+06 | 88.33 | 2.69E+09 | 8.95E+07 | 89% | 3.02E+09 | 84% |

After establishing a possible flow rate (120 mL/min) for both the fluidized bed setting and the concentration steps, five bioreactor harvests were concentrated using the kSep400. For four of these runs, the waste stream exiting the kSep chamber was regularly sampled to monitor the formation and stability of the fluidized bed (FIG. 17). A fifth run was performed, but the formation of the fluidized bed was not monitored. In all four monitored runs, the fluidized bed formed within about 8 minutes (FIG. 17). Across all 5 runs, cell recovery was >90% and any loss in viability was 1.3% (see Table 3 below). It was possible to concentrate the cells up to 2.26×10⁸ viable cells/mL. After concentration, the cells from each run were plated onto 2D and were subjected to quality testing (see discussion of FIGS. 18-21 below). In all of the kSep400 runs, at the very end of continuous concentration, a slight uptick was observed in the percent of viable cells escaping the fluidized bed (up to an additional 5%). It is unlikely that this uptick was due to exceeding chamber capacity; the same pattern observed in run 3 (˜12×10⁹ hiPSCs in the chamber) was observed in run 1 (˜3×10⁹ hiPSCs in the chamber). Without wishing to be bound by theory, it could have been due to cell settling in the feed bag, resulting in a sudden influx of concentrated cells (or cell clumps) that disturbed the fluidized bed.

TABLE 3

| | Unit | Run 1 | Run 2 | Run 3 | Run 4 | Run 5 |
|---|---|---|---|---|---|---|
| Pre-kSep VCD | v c/mL | 9.27E+05 | 1.86E+06 | 3.40E+06 | 3.30E+06 | 1.88E+06 |
| Pre-kSep Viability | % | 78.3 | 90.3 | 92.5 | 88.3 | 88.1 |
| Viable Cells into kSep | c | 3.01E+09 | 6.07E+09 | 1.19E+10 | 9.12E+09 | 4.42E+09 |
| Volume Processed | mL | 3250 | 3265 | 3500 | 2760 | 2353 |
| Post-kSep VCD | v c/mL | 9.79E+07 | 7.88E+07 | 8.52E+07 | 2.26E+08 | 1.04E+08 |
| Post-kSep Viability | % | 86.5 | 89.9 | 91.3 | 93.0 | 86.8 |
| Viable Cells Harvested | c | 2.94E+09 | 5.51E+09 | 1.08E+10 | 9.05E+09 | 4.14E+09 |
| Concentrated Volume | mL | 30 | 70 | 127 | 40 | 40 |
| Recovery | % | 97% | 91% | 91% | 99% | 94% |

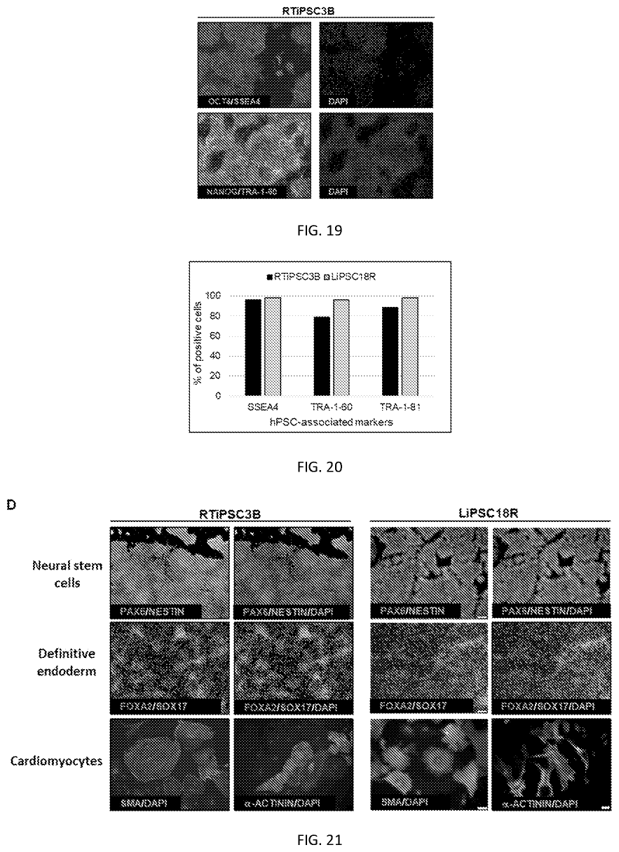

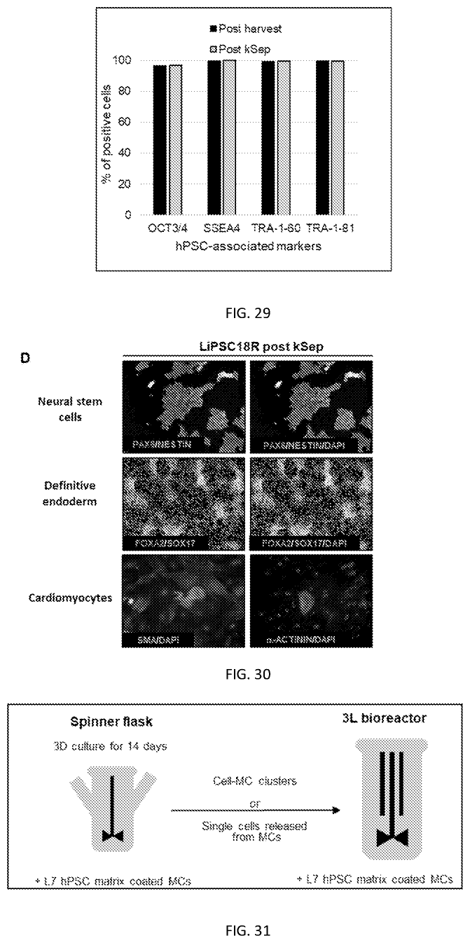

Referring to FIGS. 18-21, quality assessments of the expanded hiPSCs post kSep included cell attachment, morphology, hPSC-associated marker expression, karyotype and pluripotency. Post kSep, single cell hiPSCs (RTiPSC3B and LiPSC18R cell line) were plated in two different cell densities on 2D cell culture plates coated with L7™ hPSC Matrix. The cells, cultured in L7™ TFO2 medium, attached well and exhibited typical hPSC morphology (FIG. 18). Likewise, the cells expressed the hPSC markers as determined qualitatively by immunofluorescence staining and quantitatively by flow cytometry (FIGS. 19 and 29, respectively).

To determine whether cells concentrated by kSep are also capable of giving rise to all three germ layers, directed differentiation of RTiPSC3B and LiPSC18R cells post kSep was performed. As shown in FIG. 21, cells post expansion in a bioreactor, harvested as single cells and concentrated by kSep were capable of directly differentiating into neural stem cells, as seen by positive staining for PAX6 and NESTIN; definitive endoderm, as shown by positive staining for FOXA2 and SOX17; and cardiomyocytes, as seen by positive staining for SMA and α-ACTININ, post contraction. The karyotype of LiPSC18R from two independent bioreactor runs, followed by kSep concentration, was determined to be normal (see Table 1 above).

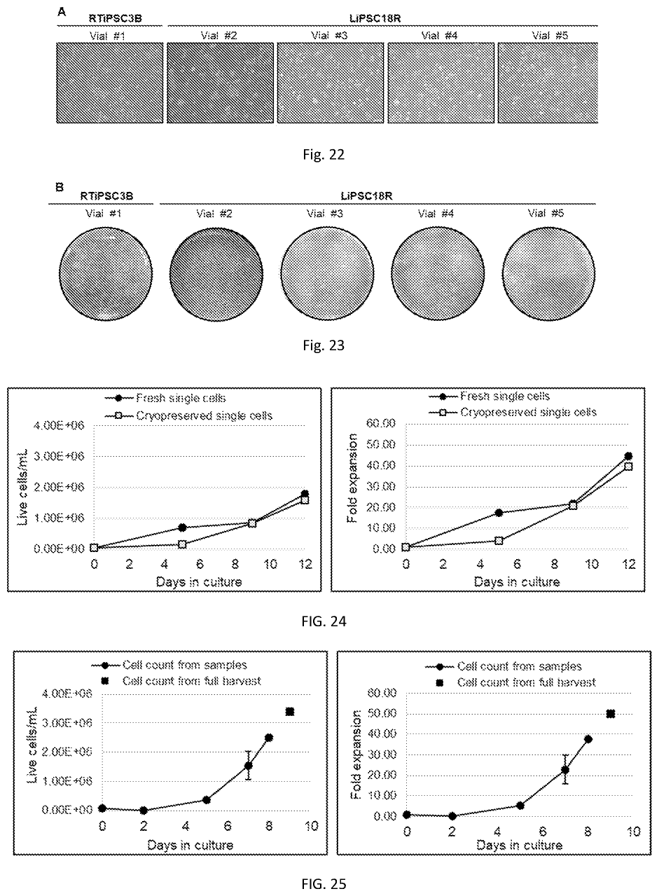

Referring next to FIGS. 22 and 23, cryopreservation of cell-based therapeutic products is a critical aspect of cell therapy. Master and working cell banks of iPSCs could be readily used for subsequent rounds of expansion and differentiation into the desired cell therapy product. A key hurdle, however, is maintaining the viability and performance of cryopreserved cells.

Human iPSCs expanded in a 3 L bioreactor and concentrated via kSep were cryopreserved in 1 mL of cryopreservation solution as described above. Cells at high viability of >85% were cryopreserved in various cell densities. The cryopreserved cells were thawed approximately two weeks after cryopreservation, and cell viability and vitality were determined. The viability of the thawed cells was similar across the various cryopreservation cell densities, however, lower than that before cryopreservation (see Table 4 below). Upon plating onto 2D cell culture plates, cells attached, expanded, had hPSC typical morphology and were positive for alkaline phosphatase staining (FIGS. 22 and 23). This data also shows the feasibility to cryopreserve hiPSCs to a high cell density of 120 and 240×10⁶ cells/ml. This will shorten a 2D seed train for further processes involving cell expansion and differentiation.

Thus, FIGS. 22 and 23 demonstrate that hiPSCs cryopreserved at high cell densities after harvest and subsequent concentration by kSep can be recovered successfully. The cells retain hPSC characteristics as shown by morphology upon plating in 2D and expression of the hPSC-associated marker, alkaline phosphatase.

TABLE 4

| | | | Cell viability (%) | |
|---|---|---|---|---|
| | | | before | Cell viability (%) |
| Vial # | Cell line | Cells/vial | cryopreservation | post thaw |
| 1 | RTiPSC3B | 4 × 10⁶ | 90 | 86 |
| 2 | LiPSC18R | 4 × 10⁶ | 87.3 | 78.2 |
| 3 | LiPSC18R | 40 × 10⁶ | 87.3 | 80.4 |
| 4 | LiPSC18R | 120 × 10⁶ | 87.3 | 79.4 |
| 5 | LiPSC18R | 240 × 10⁶ | 87.3 | 77.1 |

Next, it was examined whether the cryopreserved cells could be used to inoculate a 3 L bioreactor vessel (eliminating the need for a 2D seed train), while retaining their capability of self-renewal and differentiation. Generating sufficient cells in a 2D seed train required in order to provide adequate inoculum for a larger bioreactor is time consuming, highly manual and involves a process vulnerable to contamination. Moreover, given the variability typically observed between cell lines, culture of hPSCs by a 2D seed train depends on subjective decision making and often requires highly-trained personnel, capable of monitoring for culture irregularities, which could adversely impact subsequent cell expansion in a bioreactor. In order to overcome these challenges, the present disclosure has tested whether a 2D seed train could be avoided by thawing cryopreserved cells into a suspension culture.

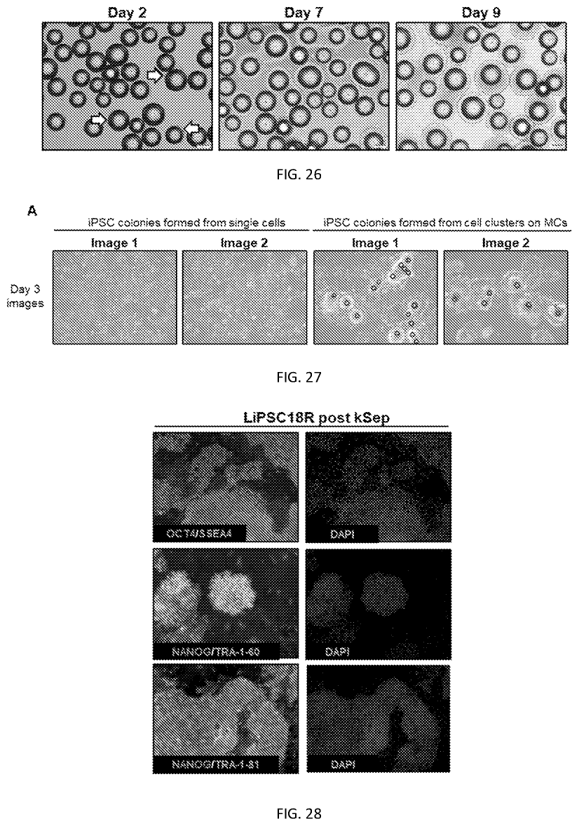

LiPSC18R, cryopreserved as single cells, were thawed and inoculated into a spinner flask at a cell density of 0.04×10⁶ cells/mL. In parallel, 2D cultured LiPSC18R cells were dissociated and inoculated as single cells into another spinner flask at the same cell density. Growth and expansion graphs demonstrate that cryopreserved and fresh single cell inoculum reach comparable cell density and fold expansion on day 9 (FIG. 24). To demonstrate scalability of these findings, a 3 L bioreactor was inoculated with LiPSC18R cells previously expanded in a 3 L bioreactor, concentrated by kSep400 and cryopreserved (0.068×10⁶ cells/mL seeding density). Nine days after inoculation, cell density of 3.5×10⁶ live cells/mL and total expansion >50-fold were achieved (FIG. 25). Representative phase contrast images show continued expansion of LiPSC18R on L7™ hPSC Matrix-coated MCs over time in suspension culture (FIG. 26). Following harvest, concentration and cryopreservation of these cells, the quality of expanded cells was assessed. FIG. 27 shows the formation of colonies with typical morphology from single cells or cell clusters expanded on MCs. Moreover, representative immunofluorescence images and flow cytometry analyses confirmed expression of hPSC-associated markers (FIGS. 28, 29, respectively). Directed differentiation of these cells to the three germ layers was also confirmed by immunostaining for cell lineage-specific markers (FIG. 30). These results demonstrate that cryopreserved hiPSC inoculum are capable of expanding in a MC-based suspension system, resulting in a large number of high-quality hiPSCs.

Particularly, cell densities of >2×10⁶ hiPSCs/mL were achieved upon inoculation of 0.02-0.07×10⁶ cells/mL in a stirred tanked bioreactor (see Table 5). As the volume of the bioreactor increases, the amount of inoculum needs to increase proportionally. Generating this inoculum in a traditional manual and open 2D process is non-ideal with increased risk of execution failures.

TABLE 5

| | | | | Inoculum cell | | |
|---|---|---|---|---|---|---|
| | Passage number | Bioreactor | | density | Harvest | Expansion |
| Cell line | at inoculation | size | Inoculum Information | cells/mL | day | fold |
| RTiPSC3B | P16 | 1 L | Cells clumps harvested from 2D | 0.04 × 10⁶ | 17 | 50 |
| RTiPSC3B | P24 | 1 L | Cells clumps harvested from 2D | 0.04 × 10⁶ | 16 | 45 |
| RTiPSC4i | P28 | 1 L | Cells clumps harvested from 2D | 0.04 × 10⁶ | 17 | 75 |
| RTiPSC3B | P22 | 3 L | Cells clumps harvested from 2D | 0.04 × 10⁶ | 15 | 160 |
| RTiPSC3B | P28 | 3 L | Cells clumps harvested from 2D | 0.04 × 10⁶ | 16 | 127 |
| RTiPSC4i | P31 | 3 L | Cells clumps harvested from 2D | 0.04 × 10⁶ | 17 | 127 |
| RTiPSC3B | P28 | 3 L | Cells clumps harvested from 2D | 0.04 × 10⁶ | 9 | 55 |
| RTiPSC3B | P28 | 3 L | Cells clumps harvested from 2D | 0.04 × 10⁶ | 12 | 66 |
| LiPSC18R | P26 | 3 L | Cells clumps harvested from 2D | 0.04 × 10⁶ | 9 | 64 |
| LiPSC18R | P29 | 3 L | Cells clumps harvested from 2D | 0.04 × 10⁶ | 11 | 44 |
| LiPSC18R | P34 | 3 L | Cells clumps harvested from 2D | 0.025 × 10⁶ | 17 | 112 |
| RTiPSC3B | P29 | 3 L | Cells clumps harvested from 2D | 0.04 × 10⁶ | 15 | 82 |
| RTiPSC4i | P30 | 3 L | Cells clumps harvested from 2D | 0.026 × 10⁶ | 11 | 72 |
| LiPSC18R | P34 | 3 L | Cryopreserved single cells | 0.04 × 10⁶ | 17 | 47 |
| LiPSC18R | P30 | 3 L | Cryopreserved single cells | 0.068 × 10⁶ | 9 | 50 |

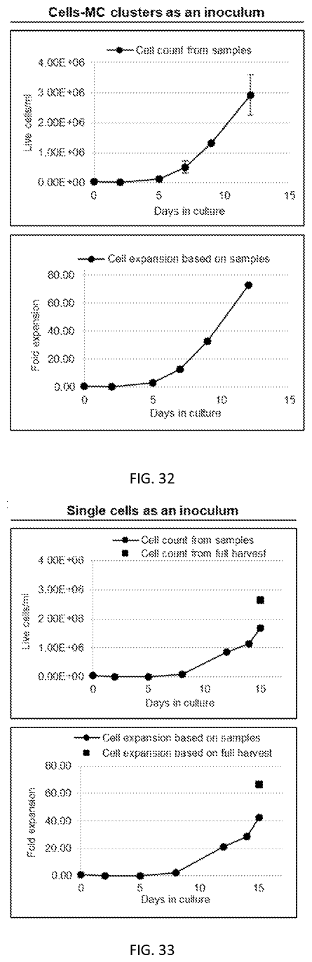

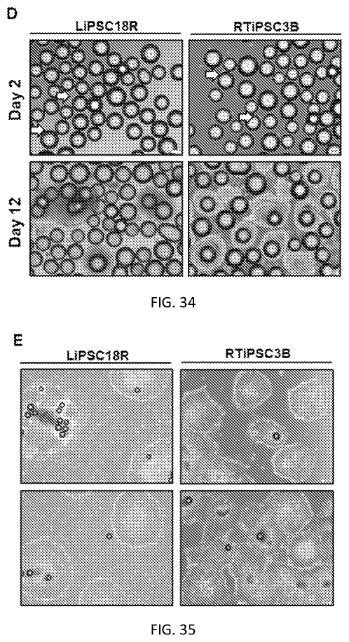

However, the present disclosure demonstrates the feasibility of inoculating 3 L bioreactors with cryopreserved cells and achieving approximately 50-fold expansion, showing that a 2D seed train could be completely replaced by a closed 3D seed train. Nevertheless, to meet the required cell number for inoculation of 50 L or larger bioreactors, a considerably sized working cell bank derived from 2D cell culture or suspension culture system is still required. To circumvent this obstacle and mitigate challenges involved with large scale manufacturing of hPSCs, the present disclosure demonstrates the feasibility of a 3D seed train. Particularly, referring to FIG. 31, two conditions were tested: re-inoculation of cell-MC clusters from a spinner flask to a 3 L bioreactor (ConLiPSC18R cells) and transfer of single cells harvested from a spinner flask to a 3 L bioreactor (RTiPSC3B cells). In both conditions, cell density at inoculation was 0.04×10⁶ cells/mL and cells were expanded with L7™ hPSC Matrix-coated MCs. The cell-MC clusters from a spinner flask resulted in a maximum cell density of 2.92×10⁶ LiPSC18R cells/mL, corresponding to >70-fold expansion in 12 days (FIG. 32), which is comparable to the results shown in FIG. 10 above. The single cells harvested from a spinner flask resulted in ˜70-fold expansion of RTiPSC3B cells on day 15, as determined by cell counts from a full harvest (FIG. 33), but, interestingly, exhibited a longer ‘lag phase’ that may be attributable to inoculating as single cells rather than cell clumps. Representative phase images of cell-MC clusters sampled from the bioreactor on day 2 and day 12 of the run, showing cell expansion (FIG. 34). FIG. 35 shows formation of colonies with typical morphology from single cells or cell clusters expanded on MCs, five days after harvest and plating onto 2D culture plates.

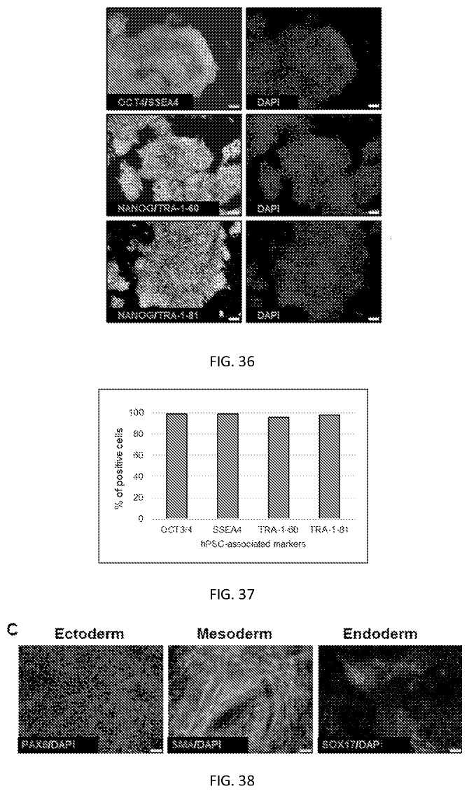

Additionally, representative immunofluorescence images (FIG. 36) showed the expression of hPSC-associated markers by RTiPSC3B cells expanded in a 3 L bioreactor through a 3D seed train (single cells harvested from a spinner flask). Flow cytometry experiments confirmed that >90% of cells expressed OCT3/4, SSEA-4, TRA-1-81 and Tra-1-60 (FIG. 37). Embryoid body formation assay showed that these cells have retained hPSC differentiation potential (FIG. 38). Based on the results described above, were able to successfully demonstrate that a 3D seed train could lead to high fold expansion of high quality cells, paving the way to commercial scale manufacturing of hPSCs.

Thus, the present disclosure has shown a microcarrier-based bioreactor suspension platform to expand hiPSCs to cell densities of >2×10⁹ cells/L using xeno-free, fully defined hPSC medium with a closed, automated process for hiPSC harvest and concentration, and extensively characterized the expanded hiPSCs. The end-to-end platform hPSC culture system, which includes the L7™ TFO2 hPSC medium and matrix, supported expansion of the hiPSCs tested in manually operated, open, spinner flasks and in automated, closed, stirred tank bioreactors. Feasibility experiments performed in spinner flasks inoculated with 0.2×10⁶ cells/mL and microcarriers resulted in 10-fold expansion within 17 days without the need for cell passaging. Results were comparable to those previously published when assessing hESC growth on laminin-coated MCs. However, as discussed herein the cells did not require adaptation to suspension culture by pre-conditioning with the MCs in static culture conditions; rather, they could be inoculated directly.

In addition to the composition of the medium in which hPSCs are expanded, optimizing cell inoculum density is a factor in hiPSC expansion in suspension systems. Particularly, when lower and higher inoculum cell densities are compared, it shows that higher fold expansion can be achieved using lower seeding densities. Moreover, seeding density not only impacts the rate and quality of expansion, but also the cost, labor and expenditure of time associated with achieving the necessary cell density at inoculation.

Furthermore, the process described herein demonstrated scalability in 1 L or 3 L bioreactor vessels. Particularly, 6-15×10⁹ cells per 3 L bioreactor run were successfully produced, (cell line and harvest day-dependent) meeting the number of cells required for a number of clinical indications. Furthermore, over 90-fold expansion was achieved within 9-16 days when using 2D cultured cells as bioreactor inoculum, which is a higher fold expansion than those achieved in spinner flask cultures. Without wishing to be bound by theory, this is likely attributable to better control of key parameters, which influence the rate of hiPSC expansion. The continuous media change, achieved through perfusion, facilitates control of key nutrients and metabolites such as glucose and lactate. pH was controlled via a one-sided control regime in which CO₂ was used to reduce pH when it drifted above the set point of 7.2. FIG. 10 shows that this prevented pH from rising more than 0.1 units above set point. However, there was no active control to raise pH (such as base addition), only the passive gradual increase in pH brought about by off-gassing of CO₂. Therefore, as cells expanded, pH dropped gradually to ˜6.8′.

Upon increase in cell expansion, however, a reduction in DO levels was observed, which could not be maintained at the set target of 50% and reached 10% at cell densities >3×10⁶ cells/mL. Previous studies have demonstrated that 02 levels regulate hPSCs metabolic flux, but the expression of pluripotency and differentiation markers in hPSCs cultured in either 20% or 5% 02 was unaltered. Moreover, no differences in proliferation were observed, suggesting that low 02 improves hPSC stemness. Others have also shown that 30% DO is the optimal condition to support hPSC expansion. In line with the present disclosure, an adverse impact on the quality of cells harvested at cell densities of ˜2.5×10⁶ cells/mL where DO levels remained >30 was not observed. Moreover, characterization of cells harvested from a 3 L bioreactor at a cell density of >5×10⁶ cells/m L, with corresponding DO levels of 10%, showed that the cells had normal karyotype, expressed hPSC-associated markers and were capable of differentiating into cells of the three germ layers. These findings suggest minimal impact of O₂ levels on the quality of cells expanded in the platform according to the present disclosure.

To increase cGMP process compliance, the present disclosure demonstrated that cells expanded in stirred tank bioreactors could be harvested and concentrated in a closed and automated manner. To date, only one other report exists of concentrating hPSCs using kSep, concentrating ˜1.2×10⁹ hPSCs tenfold, with 65% recovery of viable cells. In the process described herein, on average, the kSep process retained 94% of all harvested cells, and was able to process a 3 L bioreactor in under 30 minutes, concentrating cells up to 105-fold. Thus, the data herein shows that at least 48×10⁹ hPSCs can be harvested per kSep 400 cycle (12×10⁹ cells per chamber×four chambers), though further experiments would be needed to find the maximum capacity. This maximum capacity will be the main scale up constraint: while the 120 mL/min flow rate could reasonably process a 50 L bioreactor in 125 minutes, the total cells harvested from such a bioreactor (100×10⁹) could potentially exceed the kSep 400 capacity. This could be overcome by processing two kSep units in parallel, or by harvesting the cells from one unit over two sequential cycles.

The seamless implementation of a closed, automated concentration step using the kSep400, and subsequent recovery of high-quality hiPSCs, demonstrate the adaptability of the platform according to the present disclosure. The development of a robust, flexible and adaptable process is needed given the continued evolution of cGMP policies and innovations in large-scale manufacturing. Therefore, the MC-based and downstream processing compatible platform discussed herein allow large-scale production of hiPSCs without compromising the quality of expanded hiPSCs.

Another key element in large-scale manufacturing of cell-based therapies is maintaining the viability of cryopreserved cells such that the therapeutic potential of these cells remains intact. As shown above the cryopreserved hiPSCs produced according to the present disclosure, upon thaw, exhibited confirmed quality and viability.

In the interest of cost effectiveness and mitigation of risks such as contamination, the present disclosure has demonstrated the ability to directly inoculate cryopreserved cells into 3D. Cryopreserved cells used for the inoculum showed high fold expansion and quality, as was confirmed by cell morphology, expression of hPSC-associated markers and the ability to directly differentiate. Further development of the platform shows feasibility of a 3D seed train such that cells expanded in a 3 L bioreactor, for example, can be re-inoculated in larger bioreactor vessels. Assuming a conservative cell number of 2×10⁹ cells/L, cells from one 3 L bioreactor could be potentially serve as the inoculum for 3×50 L bioreactors. Assuming an even higher cell yield, achievable by a longer culture period in the bioreactor, one 3 L bioreactor could serve as an inoculum for several 50 L bioreactors or even one 250 L bioreactor. Together, this makes possible an expansion process which is completely 2D-free, closed, automated and requires minimal labor. This will enable commercialization of clinical indications that require large cell numbers, consequently increasing the availability of cell-based therapies.

Nonetheless, the following references an exemplary Standard Operating Procedure for the process/end-to-end platform described herein.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A process for manufacturing pluripotent stem cells, comprising
placing a plurality of microcarriers in a bioreactor;
inoculating the bioreactor with pluripotent stem cells;
incubating the pluripotent stem cells in the bioreactor for a period of time sufficient to yield a fold expansion of about 50 times or greater to give expanded pluripotent stem cells;
concentrating the expanded pluripotent stem cells; and
cryopreserving the expanded pluripotent stem cells;
wherein the pluripotent stem cells are inoculated at a seeding density of about 0.2×10⁶ cells/mL or less, and
wherein the process is a closed and/or automated process.

2. The process of claim 1, wherein the pluripotent stem cells are not passaged during incubation.

3. The process of claim 1, wherein the pluripotent stem cells used for inoculating the bioreactor are inoculated into the bioreactor as cryopreserved pluripotent stem cells.

4. The process of claim 1, wherein the pluripotent stem cells are not incubated in a 2D process prior to inoculating the bioreactor.

5. The process of claim 1, wherein the plurality of microcarriers have a particle size of about 125 μm or greater.

6. The process of claim 1, wherein the plurality of microcarriers are coated with a growth matrix prior to being placed in the bioreactor.

7. The process of claim 1, further comprising a harvesting step after incubation.

8. The process of claim 7, wherein a non-enzymatic passaging solution is used to separate the microcarriers from the expanded pluripotent stem cells.

9. The process of claim 8, wherein, after passaging with the non-enzymatic passaging solution, the pluripotent stem cells and plurality of microcarriers are run through a mesh having a mesh size sufficient to allow the pluripotent stem cells to pass through while restricting passage of the microcarriers.

10. The process of claim 9, wherein the mesh size is about 10 μm to about 100 μm.

11. The process of claim 1, wherein concentrating is performed by a continuous centrifugation device.

12. The process of claim 11, wherein a flow rate into the continuous centrifugation device is selected that allows formation of a fluidized bed in about 15 minutes or less.

13. The process of claim 12, wherein cell retention in the fluidized bed is about 80% or greater.

14. The process of claim 1, wherein cell retention after cryopreservation is about 70% or greater.

15. The process of claim 1, wherein during incubation, the microcarriers and pluripotent stem cells are subject to agitation.

16. The process of claim 15, wherein the agitation has an initial speed, and wherein the initial speed is increased after about 1 to 5 days to a second speed.

17. The process of claim 16, wherein the second speed is increased after about 1 to 5 days to a third speed.

18. The process of claim 15, wherein the agitation has an initial speed, and wherein the initial speed is increased to a second speed when the cell density reaches about 1×10⁵ cells/cm² to about 10×10⁵ cells/cm².

19. The process of claim 15, wherein, during a first 24 hours or less after inoculation, the agitation is discontinuous agitation.

20. The process of claim 19, wherein the bioreactor is a perfusion bioreactor.
